(19) United States
(12) Patent Application Publication
(10) Pub. No.: US 2014/0079632 A1
(43) Pub. Date: Mar. 20, 2014

(54) ACTIVITY-BASED PROBES FOR THE UROKINASE PLASMINOGEN ACTIVATOR

(71) Applicants: Koen Augustyns, Wilrijk (BE); Pieter Van Der Veken, Wilrijk (BE); Jonas Messagie, Wilrijk (BE); Jurgen Joossens, Wilrijk (BE); Anne-Marie Lambeir, Wilrijk (BE)

(72) Inventors: Koen Augustyns, Wilrijk (BE); Pieter Van Der Veken, Wilrijk (BE); Jonas Messagie, Wilrijk (BE); Jurgen Joossens, Wilrijk (BE); Anne-Marie Lambeir, Wilrijk (BE)

(73) Assignee: UNIVERSITEIT ANTWERPEN, Antwerpen (BE)

(21) Appl. No.: 14/115,449

(22) PCT Filed: May 9, 2012
(86) PCT No.: PCT/EP2012/058490

§ 371 Date: Nov. 4, 2013

Related U.S. Application Data
(60) Provisional application No. 61/483,787, filed on May 9, 2011.

(30) Foreign Application Priority Data
May 9, 2011 (GB) 1107649.4
Oct. 24, 2011 (GB) 1118256.5

(51) Int. Cl. A61K 49/00; C07F 9/655; C07F 5/04

(52) U.S. Cl. CPC A61K 49/0052; C07F 5/04; C07F 9/65522
USPC 424/1.77; 424/9.6; 548/110; 544/366; 548/119

(57) ABSTRACT

The present invention relates to selective trypsine-like serine protease activity-based probes, in particular urokinase plasminogen activator-activity based probes, the use thereof and methods for detecting selective urokinase activity by making use of said probes.

### FIELD OF THE INVENTION

The present invention relates to urokinase plasminogen activator (uPA)-activity-based probes, the use thereof and methods for detecting uPA activity in vitro and in vivo by making use of said probes.

### BACKGROUND TO THE INVENTION

The serine proteases of the trypsin-like (S1) family play critical roles in many key biological processes including digestion, blood coagulation, and immunity. Moreover, many S1 serine proteases are involved in diseases such as cancer¹⁻³, woundhealing^{4, 5}, arthritis⁶, skin diseases, ALS⁷, infection⁸. All the members of the S1 family have a similar protein fold with a catalytic site consisting of the oxyanion hole and the Ser, His, Asp amino acids (the catalytic triad). Typically, the members of this family have a deep S1 pocket, with at the bottom of this pocket a negatively charged Arg residue.

Many membrane-anchored serine proteases are aberrantly expressed in human cancers and have been suggested as biomarkers indicative of disease state.⁹ Evidence comes from expression studies in human cancers and in some cases from mouse models of carcinogenesis. An important trypsin-like serine protease in this context is urokinase plasminogen activator (uPA). uPA is a member of the urokinase plasminogen activator system (uPAS) and is involved in the process of cancer invasion and metastasis. uPA is synthesized and secreted as a single-chain zymogen (pro-uPA or sc-uPA), which is activated to the active two-chain form (tc-uPA) through cleavage of the Lys₁₅₈-Ile₁₅₉ peptide bond after binding to its receptor (uPAR). Activation is brought about by plasmin. When uPA is attached to its membrane-bound receptor (uPAR) it will activate plasminogen to plasmin. Plasmin plays an important role in the breakdown of the extracellular matrix (ECM). It has the ability to degrade several ECM components (e.g. fibronectin, laminin, vitronectin, type IV collagen, proteogylcans and fibrin) directly and/or through activation of certain matrix metalloproteases (MMPs). Furthermore tc-uPA, plasmin and MMPs can release/activate several mitogenic, motogenic and angiogenic growth factors, like VEGF (Vascular endothelial growth factor), HGF (hepatocyte growth factor) and TGF-β (transforming growth factor). The major endogenous inhibitor of the uPA/uPAR system is PAI-1 (plasminogen activator inhibitor 1), which will bind to the active uPA associated with his receptor, followed by the internalization of the complex.¹⁰⁻¹²

Apart from a validated target for cancer therapy and cancer biomarker¹³⁻¹⁵, uPA is also mentioned as a target and biomarker for other diseases, such as, but not limited to: arthritis¹⁶, atherosclerosis, macular degeneration¹⁷, woundhealing¹⁸, ALS⁷ and epilepsy¹⁹.

A few examples to highlight the importance of uPA in cancer:

High levels of uPA and/or PAI-1 in primary breast tumor tissues are correlated with tumor aggressiveness and poor patient outcome.²⁰ Also for other type of cancers (e.g. endometrial, prostate, colorectal) association between, uPA and PAI-1, and, tumor progression and patient outcome, has been shown. Moreover, for breast cancer the highest LOE (level of evidence) for grading clinical utility of tumor markers was achieved.²¹ Therefore uPA is a validated biomarker and visualizing/labeling uPA would be a powerful tool for diagnostics and/or therapeutic guidance.

In the above-mentioned context, it is generally recognized that a chemical tool which could visualize or capture uPA catalytic activity on a qualitative and quantitative manner in in vitro and in vivo settings will be highly useful for different non-clinical and clinical applications. Activity based probes, are specially designed chemical probes that react with mechanistically-related classes of enzymes.²² The probes typically consist of two elements: a reactive group and a tag. Additionally, some probes may contain a binding group which enhances selectivity. The reactive group usually contains an electrophile that gets covalenty-linked to a nucleophilic residue in the active site of an active enzyme. The tag may be, but is not limited to, a reporter or visualization tag such as a fluorophore, an enrichment handle such as biotin, or an alkyne or azide for introducing reporter tags, visualization tags or enrichment handles any suitable chemical reaction to obtain such tagged probes could be used such as but not limited to the Huisgen 1,3-dipolar cycloaddition (also known as click chemistry).²³

A major advantage of the activity based probes technology is the ability to monitor the availability of the enzyme active site directly, rather than being limited to protein or mRNA abundance. With classes of enzymes such as the serine proteases that often interact with endogenous inhibitors or that exist as inactive zymogens, this technique offers a valuable advantage over traditional techniques that rely on abundance rather than activity. Most importantly, in order to reach firm conclusions from experiments using the chemical probes, the latter will have to meet certain quality criteria. In a recent focus issue on chemical probes in Nature Chemical Biology (March 2010) several authors discussed these quality criteria^{24, 25}

Potency: the chemical probe needs to have a high affinity for its protein target. In case of irreversible probes, covalent binding of a probe to its target(s) should proceed sufficiently fast.

Selectivity: this is probably the most important criterion. High selectivity is absolutely essential for a chemical probe if one intends to make firm mechanistic conclusions during target profiling and target visualization experiments. Chemical proteomics is an emerging discipline essential to determine the absolute target selectivity of chemical probes.

Covalent binder: a difference between good drugs and good chemical probes is the nature of their interaction with the target. There is a strong bias in pharmaceutical industry that irreversible inhibitors would have unfavourable toxicity profiles (although numerous examples prove the contrary). For an irreversible chemical probe, the long lasting chemical knock down of a biological target will establish the relationship between a molecular target and the broader biological consequences of modulating that target. Also for imaging purposes and for MS-based chemical proteomics experiments, a strong covalent link between probe and target is an advantage.

Correlation with catalytic activity: for enzymes, chemical probes should only monitor the catalytically active fraction of the target enzyme.

Cell permeability: membrane permeability is a prerequisite for intracellular targets.

Stability: probes should have a sufficiently long half-life under the conditions that they will be used (in vitro and/or in vivo).

Versatility: ideally, the same probe can be derivatised for multiple purposes with different reporter and visualization tags.

Existing technologies to monitor proteins such as non-specific activity-based probes, peptidic probes, internally quenched fluorescent substrates and antibodies all suffer from one or more of the following disadvantages: lack of potency and selectivity, no covalent bond formation and no correlation with protein activity, low cell permeability and stability, no straightforward derivatisation with reporter or imaging tags.

In the next paragraphs we will further discuss the disadvantages associated with the current existing technologies based on non-specific activity-based probes (ABPs), activity-based probes with a peptidic structure, internally-quenched fluorescent substrates and antibodies.

Non-specific ABPs target whole enzyme families and superfamilies²². Examples are the fluorophosphonate probe tagged with rhodamine (FP-rhodamine) or with biotin (FP-biotin) developed by Cravatt et al²⁶. FP-biotin has been shown to be able to label more than 80% of the metabolically active serine hydrolases in proteomes²⁷. The major disadvantage of these probes is the lack of selectivity. The selectivity of the probe is primordial for the selective monitoring, visualization and validation of one protein target in its natural background.

The current state of the art teaches that selectivity in activity-based probes, targeting proteases, is usually obtained by introducing a peptidic part that is ideally specifically recognized by binding sites within or in the near proximity of the active site of the protease^{28, 29}. However, these peptidic compounds suffer from the disadvantages of peptides, i.e. low membrane permeability and metabolic instability.²³

Imaging of the catalytic activity of a protease has been done using internally quenched fluorescent substrates. These probes contain a matching pair of a fluorophore and a quenching unit in close proximity to each other, making the fluorophore undetectable. It is only after cleavage of the quenched substrate by a target protease, that the fluorophore becomes released from its quencher and a fluorescent signal can be detected³⁰. The group of Ralph Weissleder and colleagues has extensively explored the development of these activatable probes to image protease activity in tumours in vivo^{31, 32}. This technique has been demonstrated i.a. for cathepsin B and MMPs. The main limitations are again inherently related to the use of peptide substrates mentioned above.

Furthermore, imaging is limited to fluorescence. Perhaps, the biggest disadvantage is the fact that no covalent bond is formed with the target, resulting in rapid diffusion of the fluorophore and hence limiting its use in high resolution localization studies. Recently, in a comparative study it has been demonstrated that peptidic ABPs are superior over quenched substrate probes²³.

Finally, monoclonal antibodies are often used to demonstrate the presence of a protein target in a biological matrix. These antibodies will bind with high potency and selectivity, but they do not form a covalent bond. Depending on the epitope that is recognized, they will also detect non-catalytically active enzymes. Antibodies also hold the intrinsic disadvantages of large biomolecules mentioned above.

In view of the discussion above, we sought to develop a modular activity based probe approach that allows efficient combination of a novel, selective, non-peptidic protease binding unit with different types of visualization or reporter tags, leading to a collection of activity-based probes with an identical protease binding unit but distinct reporter or visualization tag types. Such collection could offer the possibility to quickly evaluate or even combine the results of different imaging/visualisation techniques applied to a given biological problem. The visualization/reporter tags can be different fluorophores from the visual (e.g. rhodamine) or the near-infrared region (NIR) (e.g. BODIPY), chelators for metal ions (gadolinium for MRI or ^{99m}Tc for SPECT) and ¹⁸F labelled molecules for PET. The chemical probes could be used in different applications not only limited to life sciences (e.g. imaging, histology, proteomics, . . . ) but also as tools related to quality control in any industrial process.

From the discussion above, it is clear that selectively binding, non-petidic probes containing a warhead to form a covalent bond with the target and a handle for versatile derivatisation will improve the scientific and technological state-of-the-art. To the best of our knowledge, such probes targeting uPA are not described in the scientific and patent literature.

In the next paragraphs we will describe the closest state of the art related to probes targeting the trypsin-like family and their disadvantages compared to our novel probes:

In 2008 Oikonomopoulou et al. reported a tool using a peptidic activity-based probe coupled to antibody capture.³³ The probe was built around a pro-lys peptidic fragment which was responsible for the recognition of kallikrein 6. Experts in the field will agree that the antibody approach was needed to cope with the selectivity problems related to the probe described by Oikonomopoulou et al. The probe was earlier described by Pan et al.³⁴ This article showed clearly the non-selectivity of the probes. The Ki(app) for β-tryptase, trypsin, thrombin and plasmin was in the same order of magnitude (0.6 to 6 μM). This author clearly stated that the incorporation of a proline residue increased the overall reactivity compared to single amino acid Lys probe. However, this was only a moderate activity increase with no change in the selectivity profile.

A more recent publication by Brown et al. reports the synthesis and evaluation of phosphonate-ABPs targeting matriptase, and thrombin both members of trypsin fold S1A proteases.²⁹ The authors stated that for designing broad-specificity phosphonate ABPs or specific S1A protease ABPs, a peptide sequence is required. Additionally, the leaving group of the phosphonate, peptide sequence, peptide length and peptide stability are marked as key elements for enhancing potency.²⁹ The most potent peptide containing probe has an IC₅₀ value for matriptase of 0.068 μM and a kᵢ of 490 M⁻¹ s⁻¹, which is rather modest. Moreover, the IC₅₀ values of the presented probes are obtained after a long pre-incubation period (4 hours), which emphasizes the slow reaction characteristics.

In 2011 a patent application (WO 2011/024006) around a specific group of diphenyl phosphonate probes for detection of specific proteases was available in the public domain.³⁵ However, this particular invention disclosed specific molecules with a natural amino acid diphenyl phosphonate derivative at P1 (e.g. a Valyl, Phenylalanyl, Arginyl or Lysyl group). Furthermore, all compounds disclosed in WO2011024006 contain a succinyl moiety, which is considered to be essential for said compounds. Such succinyl moiety appeared to be highly important and essential for recognition of the biomarker, selectivity of the compound towards the biomarker, improving signal to noise ratio and resistance to degradation of the compound. In fact it is not surprisingly that in general peptidyl or succinyl moieties are believed to be essential for manufacturing tagged diphenyl phosphonate probes with excellent characteristics, since even small chemical modifications to a compound can change its activity and selectivity profile unexpectedly and dramatically. Therefore peptidyl or succinyl linkers were proposed to improve the activity based properties. Given the general teaching it was therefore unexpected that tagging diphenyl phosphonate probes, without the use of peptidyl of succinyl linkers, would deliver compounds with excellent activity based properties. Nevertheless and surprisingly in view of the general teaching, the compounds disclosed in this invention were found to have excellent activity based properties, without the need of a peptidyl and/or succinyl moiety.

The most closely related application is a tool for in vivo/in vitro imaging of uPA activity using an internally quenched fluorescent substrate for uPA³⁶. The used probe has a high molecular weight, a peptide character and will release a peptide fragment which contains a fluorochrome.³⁶ Covalent labeling of the target enzyme will not be achieved. As learned from the paragraphs above this is probably not the most ideal probe for visualizing or capturing uPA activity.

The closest related state of the art describing non peptidic diphenyl phosphonates irreversible binding inhibitors reacting on a very selective and potent manner with uPA was disclosed by Joossens et al.³⁷ The described inhibitors were invented as potential drug candidates and not as imaging probes. A person skilled in the art will recognize that it is not evident to incorporate bulky substituent such as a linker combined with a visualization tag and retain at the same time the potency and selectivity.

### SUMMARY OF THE INVENTION

Based on the small irreversible uPA-inhibitors UAMC-00150³⁷ and UAMC-00251³⁷ we have designed the first activity based probes targeting selectively uPA and which show fast kinetic binding parameters and potent IC₅₀ values after a short 15 min incubation period (FIG. 1). The speed of the reaction will be beneficial for their use in preclinical and clinical settings. The selectivity was compared to 4 closely related serine proteases involved in the coagulation and fibrinolysis (tPA, thrombin, plasmin and FXa). This selectivity profile is significantly superior to the profile of the existing probes for trypsin-like serine proteases mentioned earlier and, compared to these, would make our own probes favourable for use in in vivo settings since they do not interfere with important blood coagulation enzymes. Likewise, their selectivity over tPA can be expected to also imply a cleaner profile in in vitro applications (e.g. tumor visualization in histology).

The fact that we could substitute the small groups linked to alpha amino (e.g. methyl carbamate) by a bulky substituent maintaining the same selectivity and activity towards uPA was unexpected. Replacing the methyl carbamate with a linker, in particular an azido/alkyne-alkane/polyethylene linker, has only a minor influence on activity towards uPA. Even when the bulky rhodamine is coupled to these molecules, the potency is only slightly decreased (factor 10) but maintains still an excellent selectivity profile. These probes are up till now the most potent and selective probes directed to uPA that do not interfere with proteases of the blood coagulation and fibrinolysis cascade.

The probes presented in this invention are very different from those presented in the close related publication of Brown et al²⁹:
1) The probes under this invention are targeted towards uPA a validated cancer biomarker, those of Brown et al towards matriptase (no validated biomarker status at time of our invention).
2) The herewith presented probes have faster inhibition kinetics. The highest acquired second order inhibition constant by Brown et al. was 490 M⁻¹ s⁻¹ for matriptase, while ours was ten times higher (=better) for uPA (4000 M⁻¹ s⁻¹). Important to mention that we only used a 15 min incubation period for the probe enzyme mixture prior to adding substrate in all IC₅₀-assays, while Brown et al. used 4 hours, reflecting less optimal binding kinetics.
2) As clearly and explicitly stated by the authors, the probes in the publication of Brown et al. need a peptidic tail to obtain this potency. The probe for which the protease binding moiety was directly connected to the visualization tag only obtained a second order inhibition constant of 50 M⁻¹ s⁻¹. Furthermore, the IC₅₀ value of 0.097 μM for thrombin indicates no selectivity for matriptase. In addition, based on their own expertise in the domain, the authors expect a peptidic tail to be prerequisite for obtaining acceptable binding kinetics in general with diaryl phosphonate activity-based probes. We explained in the background section that the peptidic character is disadvantageous in probe design for preclinical and clinical use.
3) The probes of Brown et al are not selective for matriptase compared to thrombin. This could have an impact on interference with the coagulation and fibrinolysis cascade. No information on the inhibition of other enzymes of this cascade is mentioned. The presented probes in this invention show at least a factor 100 selectivity toward plasmin, tPA, thrombin and FXa.
4) The presented probes by Brown et al did not show the possibility to change the visualization/reporter tag. Moreover, only biotin is presented here as an indirect visualization tag for demonstrating target enzymes via addition of [streptavidin-fluorophore]conjugates.

In general, the article of Brown et al teaches in line with other publications that a peptidic character is needed to obtain potent and selective diphenyl phosphonate probes. In our invention we have proven that it is possible to obtain non-petidic probes with all characteristics for optimal probes design: covalent binder, non petidic character, low molecular weight and the possibility to flexibly incorporate different visualization tags.

Martin et al. disclosed in 2011 an invention about diphenyl phosphonate probes.³⁵ This invention describes a methodology using specific probes with arginyl, valyl, phenylalanyl and lysyl at the P1 combined with an essential succinyl moiety. Interestingly, no data about selectivity and potency of the presented probes are described. Based on the data of Pan et al.³⁴ we could conclude that these probes are probably not potent and selective. From our investigations we know that an arginyl moiety is not the ideal P1 modification to design a selective and potent uPA probe.³⁷ In the described invention no handle is foreseen allowing flexible incorporation of different visualization tags.

The closest state to the art for selective and potent covalent uPA binding with small non-petidic molecules was published in 2007 by Joossens et al.³⁷ However, these recently disclosed molecules have no properties which make them usable as imaging probes. Moreover, It is not evident to incorporate bulky substituents and keep sufficient potency and selectivity leading to optimal probe characteristics. Most molecular probes, containing only a P1 residue and a diphenyl phosphonate warhead directly connected with a linker and visualization tag, are non selective potent covalent binders of a specific target.³⁴

Viewed from a first aspect, the invention provides a compound of Formula I or a stereoisomer, tautomer, racemic, metabolite, pro- or predrug, salt, hydrate, or solvate thereof,

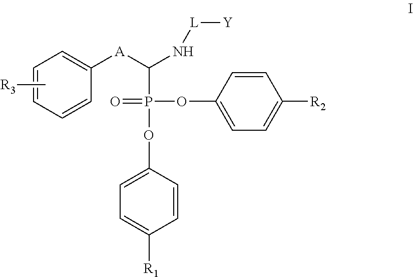

### Wherein

R₁ and R₂ are each independently selected from the group comprising —H, OH, -halo, C₁₋₆alkyl, —O—C₁₋₆alkyl, S—C₁₋₆alkyl, —NR₅R₆, —(C═O)—R₇, and SO₂—R₈;
R₅, R₆, R₉ and R₁₀ are each independently selected from the group comprising —H, —O, C₁₋₆alkyl, —O—C₁₋₆alkyl, S—C₁₋₆alkyl, and —(C═O)—C₁₋₆alkyl;
R₇ and R₈ are each independently selected from the group comprising -halo, —OH, C₁₋₆alkyl, —O—C₁₋₆alkyl, S—C₁₋₆alkyl, and —NR₉R₁₀;
R₃ is -guanidino;
A is selected from the list comprising a direct bond, and C₁₋₆alkyl;
L is selected from the list comprising: —SO₂—R₄-amide-; —SO₂—R₄-sulphonamide; —SO₂—R₄-triazole-; —SO₂—R₄-urea-; —SO₂—R₄-amine; —SO₂—R₄-carbamate-; —(C═O)—R₄-amide-; —(C═O)—R₄-sulphonamide-; —(C═O)—R₄-triazole-; —(C═O)—R₄-urea-; —(C═O)—R₄-amine-; —(C═O)—R₄-carbamate-; —(C═O)—O—R₄-amide-; —(C═O)—O—R₄-sulphonamide-; —(C═O)—O—R₄-triazole-; —(C═O)—O—R₄-urea-; —(C═O)—O—R₄-amine-; —(C═O)—O—R₄-carbamate-; —(C═O)—N—R₄-amide-, —(C═O)—N—R₄-sulphonamide-; —(C═O)—N—R₄-triazole-; —(C═O)—N—R₄-urea-; —(C═O)—N—R₄-amine-; —(C═O)—N—R₄-carbamate-; —R₄-amide-, —R₄-sulphonamide-; —R₄-triazole-; —R₄-urea-; —R₄-amine-; —R₄-carbamate-;
R₄ is selected from the list comprising —(CH₂)ₙ—, —(C₁₋₄alkyl-O)ₘ—, or —(C₁₋₄alkyl-O)ₘ—(CH₂)ₒ—;
m, n and o are each independently 1, 2, 3, 4, 5, 6, 7, 8, 9 or 10;
Y represents a detectable label.

A preferred group of compounds are those of formula I wherein:

R₁ and R₂ are each independently selected from the group comprising —H and —NH—(C═O)—CH₃;
R₃ is guanidino and wherein said R₃ is at the para position;
L is —(C═O)—O—R₄-triazole- or —(C═O)—R₄-triazole-;
R₄ is selected from the list comprising —(CH₂)ₙ—, —(C₁₋₄alkyl-O)ₘ—, or —(C₁₋₄alkyl-O)ₘ—(CH₂)ₒ—;
m is 1, 2, 3, or 4;
n is 1, 2, 3, 4, 5, 6, 7, 8, 9 or 10;
o is 1, 2, 3, or 4;
Y represents a detectable label.

The detectable label according to the invention is meant to be a group that can be instrumentally detected by a method selected from the list comprising magnetic resonance imaging, X-ray imaging, ultrasound, nuclear medicine imaging, multimodal imaging, fluorescence imaging, bioluminescence imaging, microscopy, mass detectors, wave length detectors, phosphorescent imaging, chemiluminescent imaging, . . . ,

A further aspect of this invention is to provide intermediates of formula II for preparing a compound according to formula I

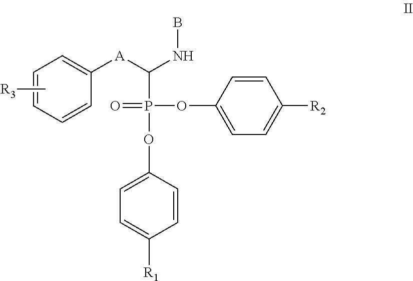

wherein
R₁ and R₂ are each independently selected from the group comprising —H, OH, -halo, C₁₋₆alkyl, —O—C₁₋₆alkyl, S—C₁₋₆alkyl, —NR₅R₆, —(C═O)—R₇, and SO₂—R₈;
R₅, R₆, R₉ and R₁₀ are each independently selected from the group comprising —H, —O, C₁₋₆alkyl, —O—C₁₋₆alkyl, S—C₁₋₆alkyl, and —(C═O)—C₁₋₆alkyl;
R₇ and R₈ are each independently selected from the group comprising -halo, —OH, C₁₋₆alkyl, —O—C₁₋₆alkyl, S—C₁₋₆alkyl, and —NR₉R₁₀;
R₃ is -guanidino;
A is selected from the list comprising a direct bond, and C₁₋₆alkyl;
B is selected from the list comprising: —(C═O)—O—R₄-alkyne, —(C═O)—O—R₄—N₃, —(C═O)—R₄-alkyne-, or —(C═O)—R₄—N₃;
R₄ is selected from the list comprising —(CH₂)ₙ—, —(C₁₋₄alkyl-O)ₘ—, or —(C₁₋₄alkyl-O)ₘ—(CH₂)ₒ—;
m, n and o are each independently 1, 2, 3, 4, 5, 6, 7, 8, 9 or 10.

A preferred group of intermediates are compounds of formula II wherein:

R₁ and R₂ are each independently selected from the group comprising —H and —NH—(C═O)—CH₃;

R₃ is guanidino and wherein said R₃ is at the para position;
B is selected from the list comprising: —(C═O)—O—R₄-alkyne, —(C═O)—O—R₄—N₃, —(C═O)—R₄-alkyne, or —(C═O)—R₄—N₃;
R₄ is selected from the list comprising —(CH₂)ₙ—, —(C₁₋₄alkyl-O)ₘ—, or —(C₁₋₄alkyl-O)ₘ—(CH₂)ₒ—;
m is 1, 2, 3, or 4;
n is 1, 2, 3, 4, 5, 6, 7, 8, 9 or 10;
o is 1, 2, 3, or 4;

In particular the compounds of this invention and compositions comprising said compounds are very useful as trypsin-like serine protease-directed activity-based probe, and may therefore in particular be used for:
detecting trypsin-like serine protease activity in a species;
analyzing the enzymatic activity of trypsin-like serine proteases in a species;
visualizing the enzymatic activity of trypsin-like serine proteases in a species;
visualizing cancer cells in a human or animal, in particular cancer cells expressing trypsin-like serine protease.
visualizing cells in a human or animal, in particular cells expressing trypsin-like serine protease

A further aspect of this invention is to provide methods for:
visualizing cancer cells in a human or animal, said method comprising administering to said human or animal, a compound or a composition according to the invention; and detecting the signal produced by the labeled compound;
visualizing an active trypsin-like serine protease in a species; said method comprising administering to said species a compound or a composition according to the invention; and detecting the signal produced by the labeled compound;
monitoring the effect of a treatment aimed at inhibiting a trypsine-like serine protease in a species; said method comprising administering to said species, at different timepoints (i.e. before, during and/or after treatment) a compound or a composition according to the invention; and detecting the signal produced by the labeled compound; wherein a reduction of the produced signal over time, is an indication that said treatment is effective.

In a specific preferred embodiment of this invention, the trypsin-like serine protease as used herein is urokinase plasminogen activator (uPA).

### BRIEF DESCRIPTION OF THE DRAWINGS

FIG. 1: Design activity based probes targeting uPA, based on irreversible inhibitors UAMC-00150/UAMC-00251

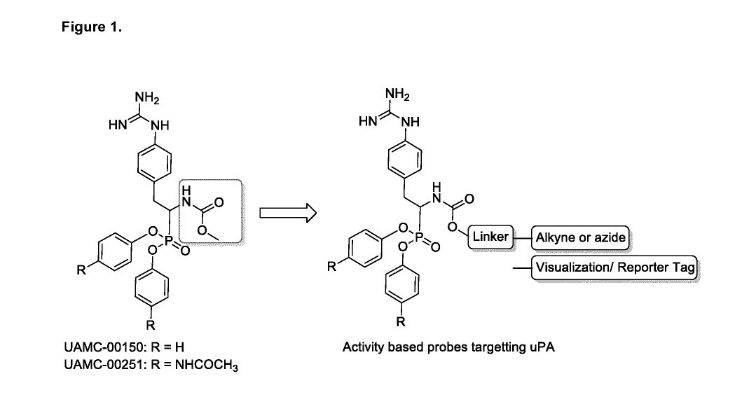

FIG. 2: General synthetic scheme to obtain the clikable and functionalized probes

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As already mentioned hereinbefore, in a first aspect the present invention provides compounds of Formula I, including a stereoisomer, tautomer, racemic, metabolite, pro- or predrug, salt, hydrate, or solvate thereof.

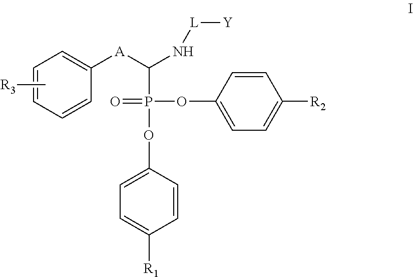

wherein
R₁ and R₂ are each independently selected from the group comprising —H, OH, -halo, C₁₋₆alkyl, —O—C₁₋₆alkyl, S—C₁₋₆alkyl, —NR₅R₆, —(C═O)—R₇, and SO₂—R₈;
R₅, R₆, R₉ and R₁₀ are each independently selected from the group comprising —H, —O, C₁₋₆alkyl, —O—C₁₋₆alkyl, S—C₁₋₆alkyl, and —(C═O)—C₁₋₆alkyl;
R₇ and R₈ are each independently selected from the group comprising -halo, —OH, C₁₋₆alkyl, —O—C₁₋₆alkyl, S—C₁₋₆alkyl, and —NR₉R₁₀;
R₃ is -guanidino;
A is selected from the list comprising a direct bond, and C₁₋₆alkyl;
L is selected from the list comprising: —SO₂—R₄-amide-; —SO₂—R₄-sulphonamide; —SO₂—R₄-triazole-; —SO₂—R₄-urea-; —SO₂—R₄-amine; —SO₂—R₄-carbamate-; —(C═O)—R₄-amide-; —(C═O)—R₄-sulphonamide-; —(C═O)—R₄-triazole-; —(C═O)—R₄-urea-; —(C═O)—R₄-amine-; —(C═O)—R₄-carbamate-; —(C═O)—O—R₄-amide-; —(C═O)—O—R₄-sulphonamide-; —(C═O)—O—R₄-triazole-; —(C═O)—O—R₄-urea-; —(C═O)—O—R₄-amine-; —(C═O)—O—R₄-carbamate-; —(C═O)—N—R₄-amide-, —(C═O)—N—R₄-sulphonamide-; —(C═O)—N—R₄-triazole-; —(C═O)—N—R₄-urea-; —(C═O)—N—R₄-amine-; —(C═O)—N—R₄-carbamate-; —R₄-amide-, —R₄-sulphonamide-; —R₄-triazole-; —R₄-urea-; —R₄-amine-; —R₄-carbamate-;
R₄ is selected from the list comprising —(CH₂)ₙ—, —(C₁₋₄alkyl-O)ₘ—, or —(C₁₋₄alkyl-O)ₘ—(CH₂)ₒ—;
m, n and o are each independently 1, 2, 3, 4, 5, 6, 7, 8, 9 or 10;
Y represents a detectable label.

When describing the compounds of the invention, the terms used are to be construed in accordance with the following definitions, unless a context dictates otherwise:

The term “alkyl” by itself or as part of another substituent refers to a fully saturated hydrocarbon of Formula CₓH₂ₓ₊₁ wherein x is a number greater than or equal to 1. Generally, alkyl groups of this invention comprise from 1 to 6 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers. C₁₋₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3-, or 4-methylcyclopentyl, cyclopentylmethylene, and cyclohexyl.

The term “halo” or “halogen” as a group or part of a group is generic for fluoro, chloro, bromo or iodo.

The term “amide” as a group or a part of a group contains a carbonyl group —(C═O)—, linked to a nitrogen atom.

The term “sulfonamide” as a group or a part of a group contains a sulfonyl group —S(═O)₂—, linked to an amine group, a divalent sulfonamide for example represents —S(═O)₂—NH—.

The term “urea” as a group or a part of a group contains 2 amine groups joined by a carbonyl (C═O) group, a divalent urea for example represents —NH—(C═O)—NH—.

The term “carbamate” as a group or a part of a group contains a carboxyl group —O—C(═O), linked to an amine group, a divalent carbamate for example represents —O—(C═O)—NH—.

The term “triazole” as a group or a part of a group refers to a five-membered ring of formula C₂H₃N₃, i.e. having 2 carbon atoms and 3 nitrogen atoms. In particular the term triazole as used herein refers to 1,2,3-triazole, more in particular the term triazole refers to the chemical moiety as shown herein below:

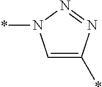 [SMILES: *c1cn(*)nn1]

The term “guanidino” as used herein refers to the chemical moiety as shown herein below:

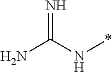 [SMILES: *NC(=N)N]

Unless a context dictates otherwise, asterisks are used herein to indicate the point at which a mono- or bivalent radical depicted is connected to the structure to which it relates and of which the radical forms part. The aforementioned graphical representation has no bearing as to the actual orientation of said groups in the remainder of the molecule.

Whenever used in the present invention, the term ‘compounds of the invention’ or a similar term is meant to include the compounds of general Formula I or II and any subgroup thereof. This term also refers to a stereoisomer, tautomer, racemic, metabolite, pro- or predrug, salt, hydrate, or solvate thereof.

As used in the specification and the appended claims, the singular forms “a”, “an” and “the” include plural referents unless the context clearly dictates otherwise. By way of example, “a compound” means one compound or more than one compound.

The ‘detectable label’ as used in the context of this invention is meant to include a signal producing group that produces an instrumentally detectable signal and may be any suitable group known in the art. In particular, the detectable label according to the invention is meant to be a group that can be instrumentally detected by a method selected from the list comprising magnetic resonance imaging, X-ray imaging, ultrasound, nuclear medicine imaging, multimodal imaging, fluorescence imaging, bioluminescence imaging, microscopy, mass detectors, wave length detectors, phosphorescent imaging, chemiluminescent imaging, . . . , The presence of said detectable label, allows for the visualization and/or detection of the compounds according to this invention. In particular the detectable label may be selected from the group comprising radio-isotopes, fluorophores, imaging agents for MRI (i.e. paramagnetic metal), X-ray responsive agents, and biotin labels or derivatives thereof.

Suitable radio-isotopes, may be selected from the list comprising ³H, ¹¹C, ¹³N, ¹⁵O, ¹⁸F, ⁵¹Cr, ⁵²Fe, ^{52m}Mn, ⁵⁵Co, ⁶⁰Cu, ⁶¹Cu, ⁶²Zn, ⁶²Cu, ⁶³Zn, ⁶⁴Cu, ⁶⁶Ga, ⁶⁷Ga, ⁶⁸Ga, ⁷⁰As, ⁷¹As, ⁷²As, ⁷⁴As, ⁷⁵Se, ⁷⁵Br ⁷⁶Br, ⁷⁷Br, ^{80m}Br, ^{82m}Br, ⁸²Rb, ⁸⁶Y, ⁸⁸Y, ⁸⁹Sr, ⁸⁹Zr, ⁹⁷Ru, ^{99m}Tc, ¹¹⁰In, ¹¹¹¹In, ^{113m}In, ^{114m}In, ^{117m}Sn, ¹²⁰I, ¹²²Xe, ¹²³I, ¹²⁴I, ¹²⁵I, ¹⁶⁶Ho, ¹⁶⁷Tm, ¹⁶⁹Yb, ^{193m}Pt, ^{195m}Pt, ²⁰¹Tl, ²⁰³Pb. In a particular embodiment the detectable labels are small sized organic PET and SPECT labels such as ¹¹C, ¹⁸F, ¹²⁴I, or ¹²⁵I. Other elements and isotopes, such as being used for therapy may also be applied. Metallic radionuclides are suitable incorporated into a chelating agent, for example by direct incorporation by methods known to the skilled artisan.

Suitable fluorophores may be selected from the non-limiting list comprising fluorescein, Alexa Fluor, Oregon Green, acridine, dansyl, NBP, BODIPY, and rhodamine,.

Imaging agents for MRI may be paramagnetic ions or superparamagnetic particles. Examples of paramagnetic ions may be selected from the group comprising Gd, Fe, Mn, Cr, Co, Ni, Cu, Pr, Nd, Yb, Tb, Dy, Ho, Er, Sm, Eu, Ti, Pa, La, Sc, V, Mo, Ru, Ce and Dy.

Suitable X-ray responsive agents include but are not limited to Iodine, Barium, Barium sulphate, Gastrofrafin, or can comprise a vesicle, liposome or polymer capsule filled with Iodine compounds and/or barium sulphate.

Although the compounds according to this invention may be synthesized in different ways, one of the preferred ways includes the use of click chemistry, in particular cycloaddition reactions involving the reaction of azides with alkyne groups. In the presence of Cu(I) salts, terminal alkynes and azides undergo 1,3-dipolar cycloaddition forming 1,4-disubstituted 1,2,3-triazoles. The choice of azides and alkynes as coupling partners is particularly advantageous as they are essentially non-reactive towards each other, in the absence of copper, and are extremely tolerant towards other functional groups and reaction conditions. As evident for a person skilled in the art, the azide may be present on the detectable label and the alkyne on the intermediate compound according to this invention; as well as vice versa. The advantage of the click-chemistry approach over conventional labelling methods is to provide the possibility to start from a single intermediate compound and to easily allow the synthesis of multiple final compounds, differing only in the used label. Furthermore, due to the selectivity of this approach, the ligation reaction can only occur at a pre-determined site in the compound, resulting in only one possible end product. Additionally, the triazole ring formed during the labelling reaction does not hydrolise and is highly stable towards oxidation and reduction, meaning that the final activity-based probes are very stable in vivo.

In an alternative embodiment the intermediates and the visualisation tags can be designed with all possible end-standing functional groups capable to form a covalent bond between the linker part and the visualisation tags. One example is an end-standing carboxylic acid and an amine which can be coupled to form an amide bond under classical reaction circumstances known to produce amide bonds. For these reaction types, leading to bond formation, procedures are well known for a person skilled in the art.

In a preferred embodiment the invention provides a compound of formula I, wherein one or more of the following restrictions apply;

R₁ and R₂ are each independently selected from the group comprising —H and —NH—(C═O)—CH₃;
R₃ is guanidino and wherein said R₃ is at the para position;
L is —(C═O)—O—R₄-triazole- or —(C═O)—R₄-triazole-;

R₄ is selected from the list comprising —(CH₂)ₙ—, —(C₁₋₄alkyl-O)ₘ—, or —(C₁₋₄alkyl-O)ₘ—(CH₂)ₒ—

m is 1, 2, 3, or 4;
n is 1, 2, 3, 4, 5, 6, 7, 8, 9 or 10;
o is 1, 2, 3, or 4;
Y represents a detectable label.

In yet a further preferred embodiment, the invention provides a compound of formula I, wherein one or more of the following restrictions apply;

R₁ and R₂ are each —H;
R₃ is guanidino and wherein said R₃ is at the para position;
L is —(C═O)—O—R₄-triazole-;

### R₄ is —(CH₂—CH₂—O)ₘ—(CH₂)—;

m is 1, 2, or 3;
Y represents a detectable label.

In yet a further preferred embodiment, the invention provides a compound of formula I, wherein one or more of the following restrictions apply;

R₁ and R₂ are each independently selected from the group comprising —H and —NH—(C═O)—CH₃;
R₃ is guanidino and wherein said R₃ is at the para position;
L is —(C═O)—O—CH₂—CH₂—CH₂-triazole-;
Y represents a detectable label.

In a further aspect, the present invention provides an intermediate of formula II for preparing a compound according to this invention,

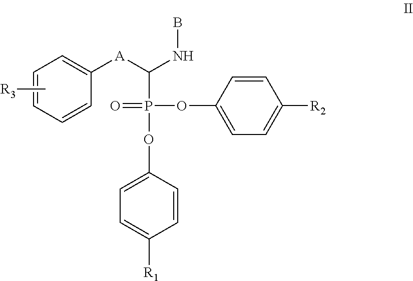

wherein
R₁ and R₂ are each independently selected from the group comprising —H, OH, -halo, C₁₋₆alkyl, —O—C₁₋₆alkyl, S—C₁₋₆alkyl, —NR₅R₆, —(C═O)—R₇, and SO₂—R₈;
R₅, R₆, R₉ and R₁₀ are each independently selected from the group comprising —H, —O, C₁₋₆alkyl, —O—C₁₋₆alkyl, S—C₁₋₆alkyl, and —(C═O)—C₁₋₆alkyl;
R₇ and R₈ are each independently selected from the group comprising -halo, —OH, C₁₋₆alkyl, —O—C₁₋₆alkyl, S—C₁₋₆alkyl, and —NR₉R₁₀;
R₃ is -guanidino;
A is selected from the list comprising a direct bond, and C₁₋₆alkyl;
B is selected from the list comprising: —(C═O)—O—R₄-alkyne, —(C═O)—O—R₄—N₃, —(C═O)—R₄-alkyne, or —(C═O)—R₄—N₃
R₄ is selected from the list comprising —(CH₂)ₙ—, —(C₁₋₄alkyl-O)ₘ—, or —(C₁₋₄alkyl-O)ₘ—(CH₂)ₒ—;
m, n and o are each independently 1, 2, 3, 4, 5, 6, 7, 8, 9 or 10.

A preferred group of intermediates are those of formula II, wherein one or more of the following restrictions apply;

R₁ and R₂ are each independently selected from the group comprising —H and —NH—(C═O)—CH₃;
R₃ is guanidino and wherein said R₃ is at the para position;
B is selected from the list comprising: —(C═O)—O—R₄-alkyne, —(C═O)—O—R₄—N₃, —(C═O)—R₄-alkyne, or —(C═O)—R₄—N₃
R₄ is selected from the list comprising —(CH₂)ₙ—, —(C₁₋₄alkyl-O)ₘ—, or —(C₁₋₄alkyl-O)ₘ—(CH₂)ₒ—;
m is 1, 2, 3, or 4;
n is 1, 2, 3, 4, 5, 6, 7, 8, 9 or 10;
o is 1, 2, 3, or 4;

A further preferred group of intermediates are those of formula II, wherein one or more of the following restrictions apply;

R₁ and R₂ are each —H;
R₃ is guanidino and wherein said R₃ is at the para position;
B is selected from the list comprising: —(C═O)—O—R₄-alkyne, and —(C═O)—O—R₄—N₃,
R₄ is selected from the list comprising —(CH₂—CH₂—O)ₘ—(CH₂)— or —(CH₂)ₙ—;
m is 1, 2 or 3;
n is 6

A further preferred group of intermediates are those of formula II, wherein one or more of the following restrictions apply;

R₁ and R₂ are each independently selected from the group comprising —H and —NH—(C═O)—CH₃;
R₃ is guanidino and wherein said R₃ is at the para position;
B is selected from the list comprising —(C═O)—O—CH₂—CH₂—CH₂—N₃ and —(C═O)—O—CH₂—CH₂—CH₂-alkyne;

As a further object, this invention provides a composition comprising a compound according to this invention. It is evident for a person skilled in the art that the formulation of said composition will depend on the type of detectable label used.

For example, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

Generally, the compounds of this invention may be formulated as a pharmaceutical preparation or pharmaceutical composition comprising at least one compound of the invention and at least one pharmaceutically acceptable carrier, diluent or excipient and/or adjuvant.

By means of non-limiting examples, such a formulation may be in a form suitable for oral administration, parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives.

In addition, co-solvents such as alcohols may improve the solubility and/or the stability of the compounds. In the preparation of aqueous compositions, addition of salts of the compounds of the invention can be more suitable due to their increased water solubility.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, ocular, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formula I or II, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778,U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

It is an object of the present invention to provide compounds and/or compositions according to this invention, for multiple purposes including, but not limited to:
detecting trypsin-like serine protease activity in a species.
analyzing the enzymatic activity of trypsin-like serine proteases in a species.
visualizing the enzymatic activity of trypsin-like serine proteases in a species.
visualizing cancer cells in a human or animal, in particular in cancer cells expressing a trypsine-like serine protease

As used herein, the term “species” is meant to include protein containing material, cell lysates, cells, tissue lysates, tissues, animals and humans.

As used herein, the term “serine protease” is meant to include a protease (enzyme that cuts peptide bonds in proteins) in which at least one of the amino acids in the active site of the enzyme is serine. It is generally known that serine proteases can be divided into families based on their structural homology, and subsequently in subgroups based on sequence similarities, such as for example the group of (chymo)trypsin-like proteases.

In a preferred embodiment the trypsin-like serine protease is urokinase plasminogen activator (uPA), also known as urokinase. This is a protease that is expressed at several physiological locations, such as in the blood or the extracellular matrix. Its primary substrate is plasminogen, which is an inactive form of the serine protease plasmin. Due to its role in thrombolysis and extracellular matrix degradation, uPA is believed to be involved in vascular diseases and cancer. The compounds and compositions according to this invention are therefore very suitable in detecting, analyzing and visualizing cancer cells expressing uPA.

A further aspect of this invention is to provide methods for:
visualizing cancer cells in a human or animal, said method comprising administering to said human or animal, a compound or a composition according to the invention; and detecting the signal produced by the labeled compound;
visualizing an active trypsin-like serine protease in a species; said method comprising administering to said species a compound or a composition according to the invention; and detecting the signal produced by the labeled compound;
monitoring the effect of a treatment aimed at inhibiting a trypsin-like serine protease in a species; said method comprising administering to said species, at different timepoints (i.e. before, during and/or after treatment) a compound or a composition according to the invention; and detecting the signal produced by the labeled compound; wherein a reduction of the produced signal over time, is an indication that said treatment is effective.

The compounds of the present invention can be prepared according to the reaction schemes provided in the examples hereinafter, but those skilled in the art will appreciate that these are only illustrative for the invention and that the compounds of this invention can be prepared by any of several standard synthetic processes commonly used by those skilled in the art of organic chemistry.

The invention will now be illustrated by means of the following synthetic and biological examples, which do not limit the scope of the invention in any way.

### Examples

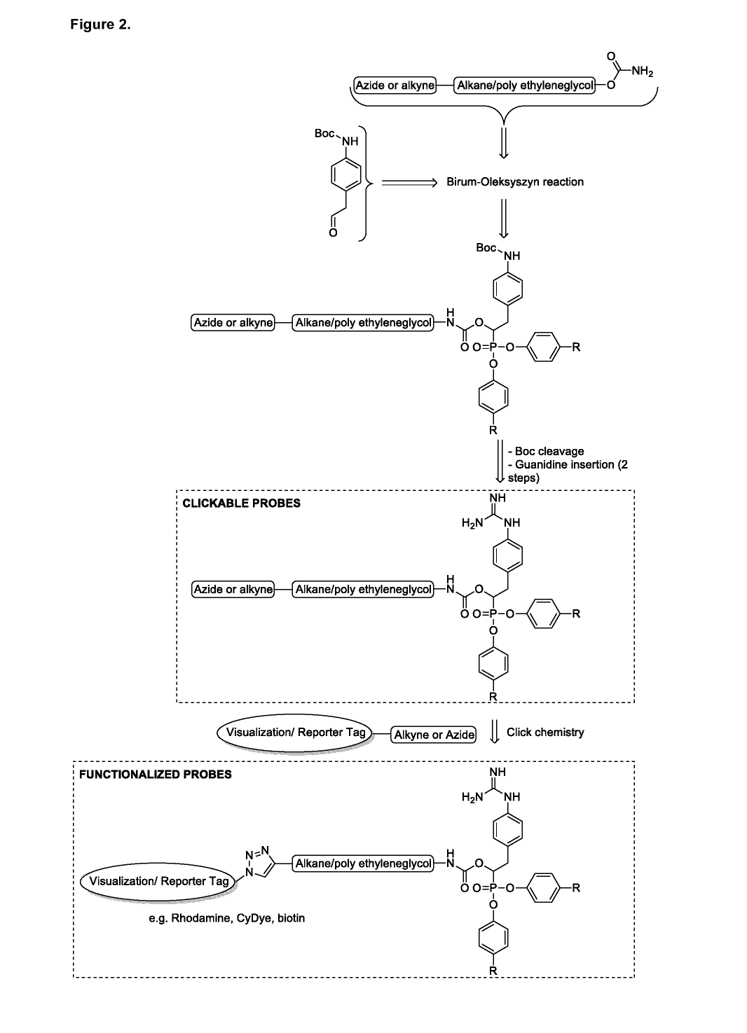

FIG. 2 outlines the general synthetic strategy to obtain the clickable and functionalized probes. The key reaction for synthesizing the probes is the Birum-Oleksyszyn reaction. This reaction requires an aldehyde, a carbamate, a phosphite and a catalyst. If a lewis acid as catalyst is used, the molecule can contain an acid labile Boc-protecting group.³⁸ Using different carbamates in this reaction gives us the opportunity to introduce different side chains, while the benzylguanidine-group is maintained. When the carbamate-based intermediates contain an azide or alkyne functional group, the obtained probes (clickable probes) can be conjugated to different visualization tags (e.g. rhodamine, biotin). This leads to functionalized probes, which can be used for labeling and visualizing uPA.

Scheme 1 shows the synthesis of the different carbamates. Trichloroacetyl isocyanate is used as reagent to convert the alcohols (intermediates 4-6, 10, 13, 17, 18, 23) into the corresponding carbamates (intermediates 7-9, 11, 14, 19, 20, 24). These alcohols were bought as such, or were made from commercially available ethylene glycols and halogenated alkylalcohols via nucleophilic substitution reactions.

Boc-protected 4-aminophenylacetaldehyde (intermediate 27), prepared from the corresponding Boc protected alcohol (intermediate 26) with Dess-Martin periodane (Scheme 2) was used, together with the different carbamates, in the amidoalkylation reaction to yield diarylphosphonates (intermediates 28-36). After acidolytic removal of the Boc protecting group (intermediates 37-45), N,N′-bis(tert-butoxycarbonyl)-1-guanylpyrazole was used to introduce the protected guanidine group (intermediates 46-54). Boc deprotection afforded intermediates 55-63.

Scheme three shows the synthesis of different visualization tags (rhodamine, biotin, BODIPY and 4-fluorobenzamide), which are used as exemplary visualization tags and show the conjugation possibilities of the clickable probes.

Piperazine rhodamine (intermediate 67) was synthesized via a known literature procedure.³⁹ Although the authors mention that work up with flash chromatography is not necessary, we did not manage to get the compound pure without flash chromatography. Coupling with 8-azidooctanoic acid (intermediate 65) yielded azido-rhodamine (intermediate 68).

Reaction of biotin with DCC and N-hydroxysuccinimide yields the activated biotin-ester (intermediate 71) which can easily react with 2-(2-(2-(2-azidoethoxy)ethoxy)ethoxy)ethanamine to azido-biotin (intermediate 72).

Condensation of two 2,4-dimethyl-pyrrole units, an acylchloride (intermediate 69) and BF₃ gives the desired azido-BODIPY (intermediate 73).

A fifth possible label to synthesize is Cy5. A synthesis is described by Jung et al and can be further modified with an azide linker. (Michael E. Jung and Wan-Joong Kim, Bioorganic & Medicinal Chemistry, 2006, 14, 92-97)

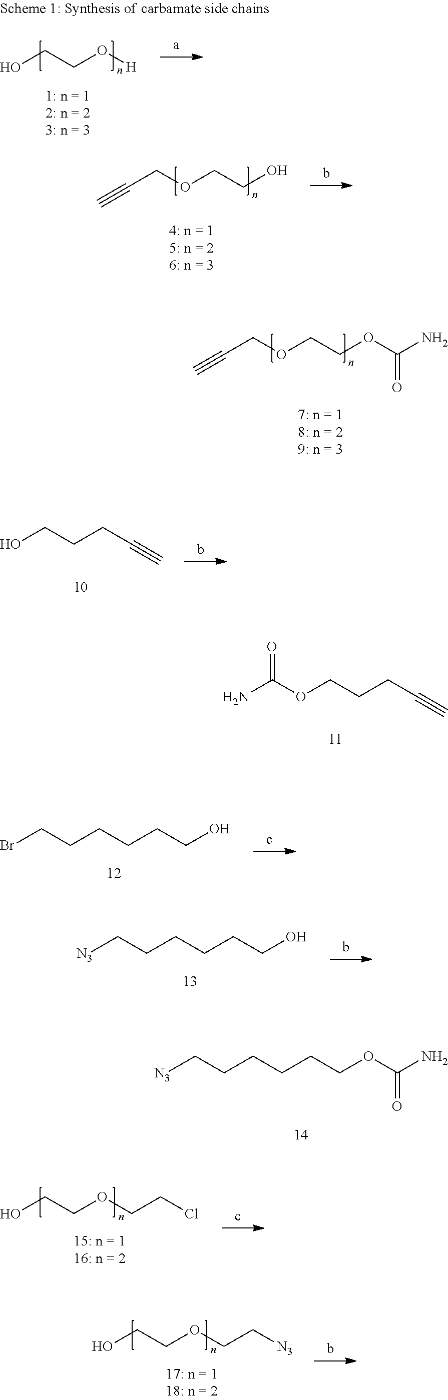

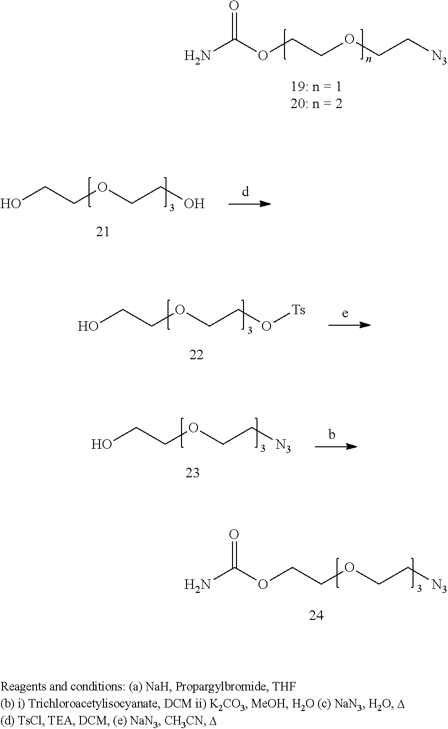

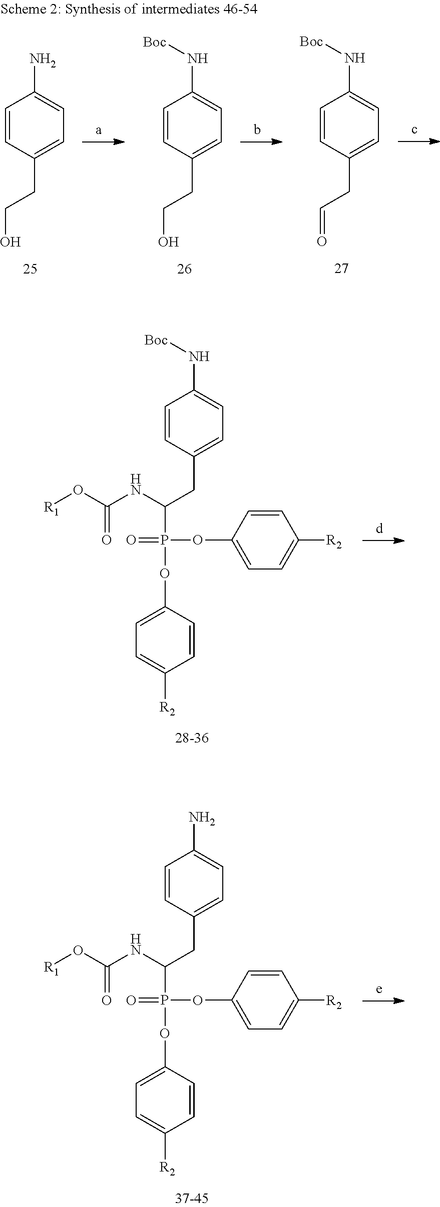

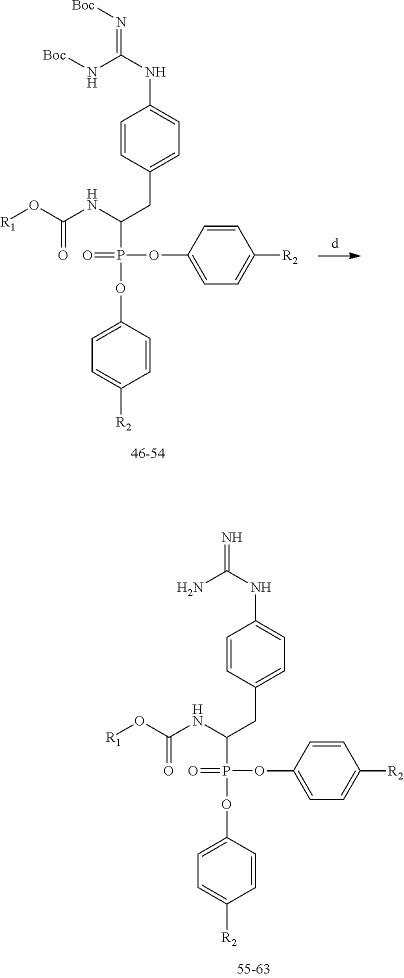

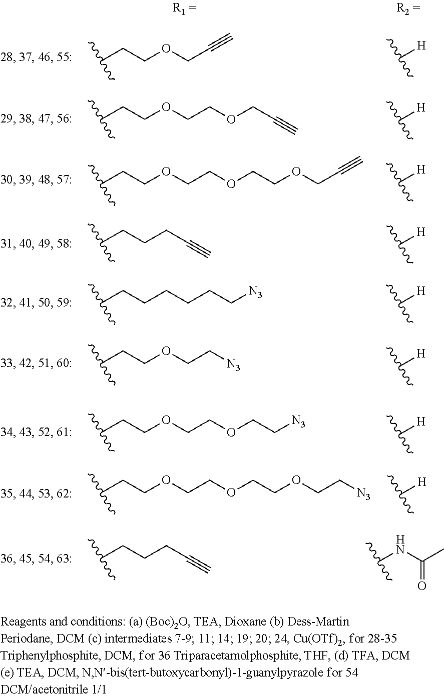

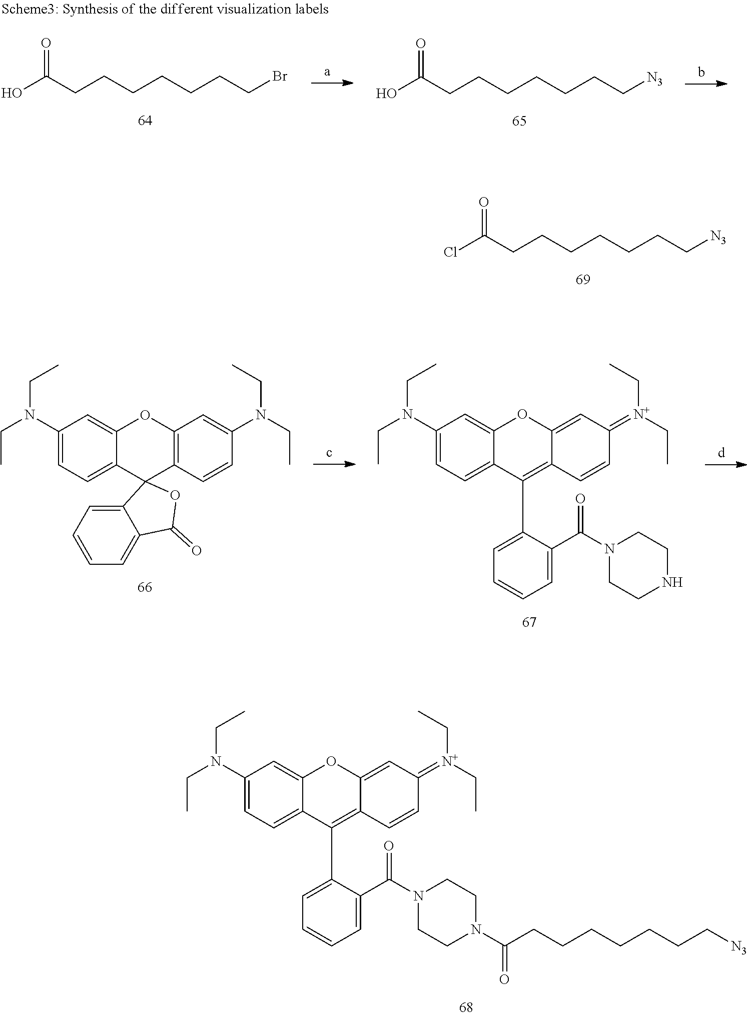 [SMILES: O=C(O)CCCCCCCBr ; [N-]=[N+]=NCCCCCCCC(=O)O ; [N-]=[N+]=NCCCCCCCC(=O)Cl ; CCN(CC)c1ccc2c(c1)Oc1cc(N(CC)CC)ccc1C21OC(=O)c2ccccc21 ; CCN(CC)c1ccc2c(-c3ccccc3C(=O)N3CCNCC3)c3ccc(=[N+](CC)CC)cc-3oc2c1 ; CCN(CC)c1ccc2c(-c3ccccc3C(=O)N3CCN(C(=O)CCCCCCCN=[N+]=[N-])CC3)c3ccc(=[N+](CC)CC)cc-3oc2c1]

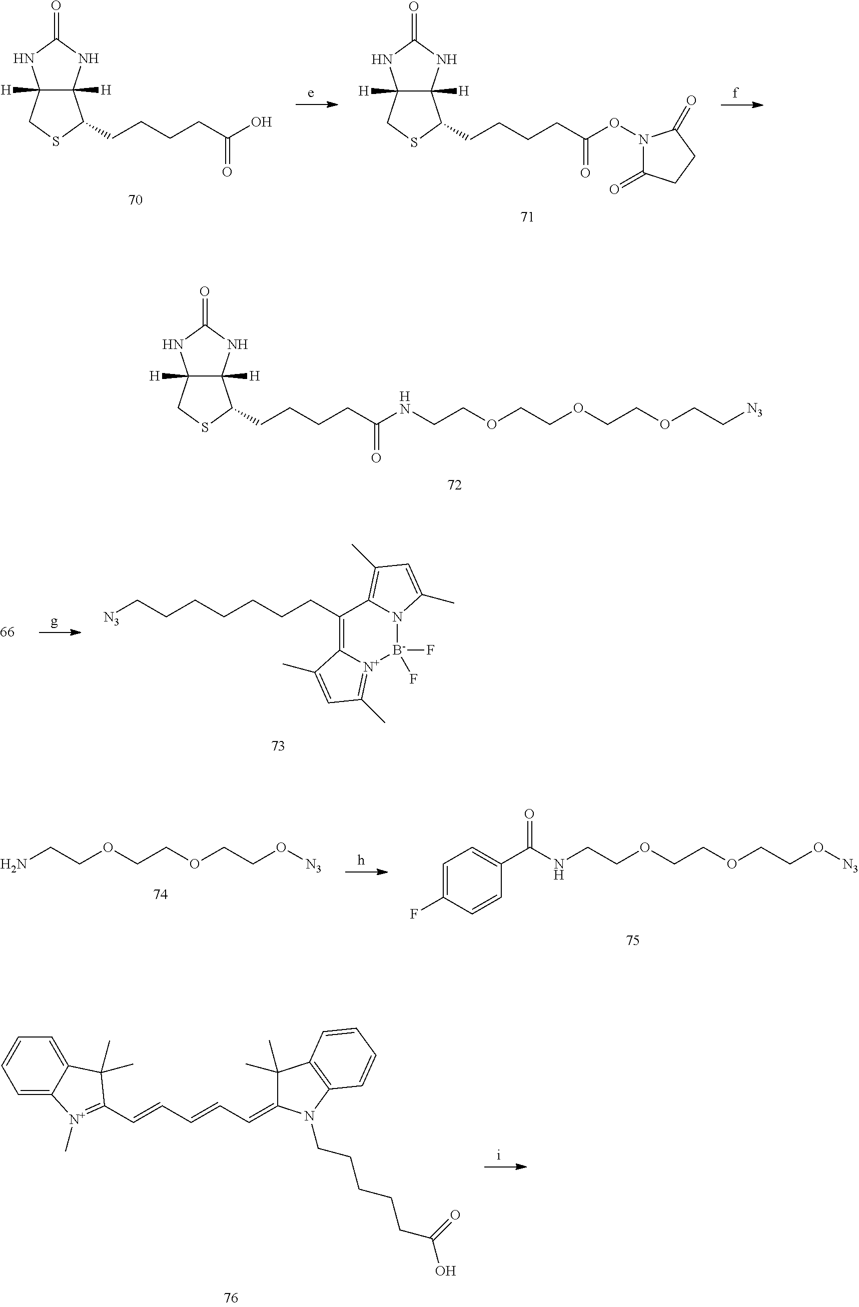

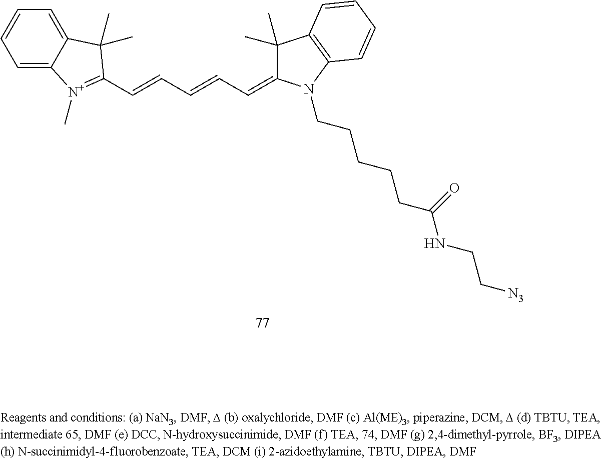 [SMILES: C[N+]1=C(/C=C/C=C/C=C2/N(CCCCCC(=O)NCCN=[N+]=[N-])c3ccccc3C2(C)C)C(C)(C)c2ccccc21]

### Experimental Section

Reagents were obtained from Sigma-Aldrich, Acros or Fluorochem. Characterization of all compounds was done with ¹H NMR and mass spectrometry. ¹H NMR spectra were recorded on a 400 MHz Bruker Avance III nanobay spectrometer. ES mass spectra were obtained from an Esquire 3000plus iontrap mass spectrometer from Bruker Daltonics. Purity was verified using one of the following methods: HPLC systems using, a mass or UV-detector. Water (A) and CH₃CN (B) were used as eluents. LC-MS spectra were recorded on an Agilent 1100 Series HPLC system using a Alltech Prevail C18 column (2.1×50 mm, 3 μm) coupled with an Esquire 3000plus as MS detector and a 5-100% B, 20 min gradient was used with a flow rate from 0.2 mL/min. Formic acid 0.1% was added to solvents A and B. Reversed phase HPLC was run on a Gilson instrument equipped with an Ultrasphere ODS column (4.6×250 mm, 5 μm). A 10-100% B, 35 min gradient was used with a flow rate from 1 mL/min. Trifluoroacetic acid 0.1% was added to solvent A and B. A wavelength of 214 nm was used. When necessary, the products were purified with flash chromatography on a Flashmaster II (Jones chromatography).

### Synthesis of Intermediates

### Reaction Procedure A

### Intermediate 4: 2-(prop-2-ynyloxy)ethanol

Ethane-1,2-diol (182 mmol) in THF (100 ml) was added dropwise to a solution of sodium hydride (45.9 mmol) in THF (80 ml) at 0° C. during 30 minutes. The solution was stirred for 2 hours at room 15 temperature. 3-bromoprop-1-yne (41.8 mmol) was added and the solution was refluxed overnight, followed by addition of water (80 ml). Solvent was evaporated and extracted with EtOAc (4×100 ml). The combined organic layers were washed with brine, dried over anhydrous Na₂SO₄, filtered and concentrated in vacuo. The obtained mixture was purified with flash chromatography (100% Hexane to 40% EtOAc in Hexane)

Yield: 40%

MS (ESI) m/z 123 [M+Na]⁺

¹H-NMR (CDCl₃, 400 MHz) δ1.9 (br s, 1H), 2.44 (t, J=2.4 Hz, 1H), 3.64 (t, J=4.4 Hz, 2H), 3.76 (t, J=4.4 Hz, 2H), 4.20 (d, J=2.4 Hz, 2H)

The following intermediates were prepared in a similar way:

### Intermediate 5: 2-(2-(prop-2-ynyloxy)ethoxy)ethanol

Yield: 36%

MS (ESI) m/z 167 [M+Na]⁺

¹H-NMR (CDCl₃, 400 MHz) δ 2.44 (t, J=2.4 Hz, 1H), 3.62 (t, J=4.4 Hz, 2H), 3.69-3.77 (m, 6H), 4.22 (d, J=2.4 Hz, 2H)

### Intermediate 6: 2-(2-(2-(prop-2-ynyloxy)ethoxy)ethoxy)ethanol

Yield: 41%

MS (ESI) m/z 211.0 [M+Na]+

¹H-NMR (CDCl₃, 400 MHz) δ 2.28 (br s, 1H), 2.44 (t, J=2.4 Hz, 1H), 3.62-3.75 (m, 12H), 4.21 (d, J=2 Hz, 2H)

### Reaction Procedure B

### Intermediate 7: 2-(prop-2-ynyloxy)ethyl carbamate

To a solution of 2-(prop-2-ynyloxy)ethanol (intermediate 4) (8.99 mmol) in dry DCM (50 ml), was added 2,2,2-trichloroacetyl isocanate (10.79 mmol) at 0° C. After 1 hour stirring at room temperature, the solvent was evaporated and the reaction mixture was dissolved in 30 ml MeOH en 3 ml Water. K₂CO₃ (15.46 mmol) was added and the reaction was allowed to stir overnight. MeOH was evaporated and water (50 ml) was added. This water layer was extracted twice with EtOAc. The organic layers were combined, washed with brine, dried over anhydrous Na₂SO₄, filtered and evaporated. A yellow oily liquid was obtained.

Yield: 79%

MS (ESI) m/z 182 [M+K]⁺

¹H-NMR (CDCl₃, 400 MHz) δ 2.45 (t, J=2.4 Hz, 1H), 3.75 (t, J=4.4 Hz, 2H), 4.21 (d, J=2 Hz, 2H), 4.26 (t, J=4.4 Hz, 2H), 4.79 (br s, 2H)

The following intermediates were prepared in a similar way:

### Intermediate 8: 2-(2-(prop-2-ynyloxy)ethoxy)ethylcarbamate

Yield: 74%

MS (ESI) m/z 226 [M+K]⁺

¹H-NMR (CDCl₃, 400 MHz) δ 2.40 (t, J=2.4 Hz, 1H), 3.65 (m, 6H), 4.20 (m, 4H), 4.90 (br s, 2H)

### Intermediate 9: 2-(2-(2-(prop-2-ynyloxy)ethoxy)ethoxy)ethylcarbamate

Yield: 84%

¹H-NMR (CDCl₃, 400 MHz): δ 2.44 (t, J=2.4 Hz, 1H), 3.68 (m, 10H), 4.22 (m, 4H), 4.95 (br s, 2H)

### Intermediate 11: pent-4-ynyl carbamate

Yield: 65%

¹H-NMR (400 MHz, CDCl₃): δ 1.87 (q, J=6.8 Hz, 2H), 1.98 (t, J=2.8 Hz, 1H), 2.3 (dt, J=2.8 Hz and J=6.8 Hz, 2H), 4.23 (t, J=6.4 Hz, 2H), 10.82 (br s, 2H)

### Intermediate 14: 6-azidohexyl carbamate

Yield: 68%

MS (ESI) m/z 209.2 [M+Na]⁺

¹H-NMR (CDCl₃, 400 MHz): δ 1.39 (m, 4H), 1.63 (m, 4H), 3.27 (t, J=6.8 Hz, 2H), 4.06 (t, J=6.8 Hz, 2H), 4.59 (br s, 2H)

### Intermediate 19: 2-(2-azidoethoxy)ethylcarbamate

Yield: 88%

MS (ESI) m/z 197.1 [M+Na]⁺

¹H-NMR (CDCl₃, 400 MHz): δ 3.39 (t, J=4.8 Hz, 2H), 3.69 (m, 4H), 4.24 (t, J=4.4 Hz, 2H), 4.79 (br s, 2H)

### Intermediate 20: 2-(2-(2-azidoethoxy)ethoxy)ethylcarbamate

Yield: 80%

MS (ESI) m/z 241.2 [M+Na]⁺, 257.1 [M+K]⁺

¹H-NMR (CDCl₃, 400 MHz): δ 3.40 (t, J=5.2 Hz, 2H), 3.70 (m, 8H), 4.23 (m, 2H), 4.82 (br s, 2H)

### Intermediate 24: 2-(2-(2-(2-azidoethoxy)ethoxy)ethoxy)ethylcarbamate

Yield: 70%

MS (ESI) m/z 285.2 [M+Na]⁺

¹H-NMR ((CDCl₃, 400 MHz): δ 3.39 (t, J=5.2 Hz, 2H), 3.67 (m, 12H), 4.23 (m, 2H), 4.81 (br s, 2H)

### Reaction Procedure C

### Intermediate 13: 6-azidohexan-1-ol

To a solution of 6-bromohexan-1-ol (intermediate 12) (5.52 mmol) in Water (10 ml), was added sodium azide (27.6 mmol) and stirred at 90° C. for 3 h. The mixture was cooled to RT and treated with 2N HCl. The aqueous solution was extracted (3×) with EtOAc, brine and dried over Na₂SO₄. The solvent was removed in vacuo to give a slight yellow oil which was used in the next step without further purification.

Yield: 81%

¹H-NMR (CDCl₃, 400 MHz): δ 1.41 (m, 6H), 1.61 (m, 4H), 3.27 (t, J=6.8 Hz, 2H), 3.65 (t, J=6.4 Hz, 2H)

The following intermediates were prepared in a similar way:

### Intermediate 17: 2-(2-azidoethoxy)ethanol

Yield: 80%

MS (ESI) m/z 154.1 [M+Na]⁺

¹H-NMR (CDCl₃, 400 MHz): δ 2.27 (br s, 1H), 3.41 (t, J=5.2 Hz, 2H), 3.62 (t, J=4.8 Hz, 2H), 3.70 (t, J=4.8 Hz, 2H), 3.76 (t, J=4.8 Hz, 2H)

### Intermediate 18: 2-(2-(2-azidoethoxy)ethoxy)ethanol

Yield: 82%

MS (ESI) m/z 198.1 [M+Na]⁺

¹H-NMR (CDCl₃, 400 MHz): δ 2.36 (t, J=6 Hz, 1H), 3.40 (t, J=5.2 Hz, 2H), 3.62 (t, J=4.4 Hz, 2H), 3.68 (m, 6H), 3.73 (m, 2H)

### Reaction Procedure D:

### Intermediate 22: 2-(2-(2-(2-hydroxyethoxy)ethoxy)ethoxy)ethyl 4-methylbenzenesulfonate

2,2′-(2,2′-oxybis(ethane-2,1-diyl)bis(oxy))diethanol (intermediate 21) (40.0 mmol) and TEA (20.00 mmol) were dissolved in DCM (50 ml). Then, Tosyl-Cl (1.907 g, 10.00 mmol) was added in one portion. The resulting mixture was stirred for one hour at room temperature. The mixture was washed with 1M KHSO₄ and 5% NaHCO₃, dried over Na₂SO₄ and filtered. After evaporation of the solvent a yellow oil was formed.

Yield: 72%

MS (ESI) m/z 371.3 [M+Na]⁺

¹H-NMR (CDCl₃, 400 MHz): δ 2.34 (br s, 1H), 2.45 (s, 3H), 3.55-3.73 (m, 14H), 4.17 (t, 2H), 7.33 (d, J=8.4 Hz, 2H), 7.80 (d, J=8.4 Hz, 2H)

### Reaction Procedure E

### Intermediate 23: 2-(2-(2-(2-azidoethoxy)ethoxy)ethoxy)ethanol

To a solution of 2-(2-(2-(2-hydroxyethoxy)ethoxy)ethoxy)ethyl methanesulfonate (intermediate 22) (1.836 mmol) in acetonitrile (10 ml) was added sodium azide (9.18 mmol) and was refluxed for 23 hours. The solvent was evaporated, EtOAc (50 ml) and water (50 ml) were added. The organic layers were combined, washed with 2N HCl and brine, and dried over Na₂SO₄. The solvent was removed in vacuo to yield intermediate 23 as an oily product.

Yield: 77%

MS (ESI) m/z 242.2 [M+Na]⁺

¹H-NMR (CDCl₃, 400 MHz): δ 2.25 (br s, 1H), 3.39 (t, J=5.2 Hz, 2H), 3.61 (t, J=4.8 Hz, 2H), 3.68 (m, 10H), 3.73 (t, J=4.8 Hz, 2H)

### Reaction Procedure F

### Intermediate 26: tert-butyl 4-(2-hydroxyethyl)phenylcarbamate

To a solution of 2-(4-aminophenyl)ethanol (intermediate 25) (87 mmol) in 100 ml Dioxane were added triethylamine (87 mmol) and Boc₂O (96 mmol). The mixture was stirred overnight at RT. The solution was concentrated in vacuo, dissolved in EtOAc, washed with 2N HCl, saturated NaHCO₃ and brine solution. The organic layer was dried over Na₂SO₄. Purification was obtained by flash chromatography (100% Hexane to 100% EtOAc).

Yield: 50%

MS (ESI) m/z 260 [M+Na]⁺, 275.90 [M+K]⁺

1H-NMR (CDCl₃, 400 MHz): δ 1.5 (s, 9H), 2.83 (t, J=6.4 Hz, 2H), 3.83 (t, J=6.4 Hz, 2H), 6.4 (s, 1H), 7.15 (d, J=8.4 Hz, 2H), 7.30 (d, J=8.4 Hz, 2H)

### Reaction Procedure G

### Intermediate 27: tert-butyl 4-(2-oxoethyl)phenylcarbamate

To a stirred solution of Dess-MartinPeriodinane (31.0 mmol) in DCM (50 ml), tert-butyl 4-(2-hydroxyethyl)phenylcarbamate (intermediate 26) (20.65 mmol) was added. The solution was stirred at RT for 4 h. The resulting solution was poured into a vigorously stirred saturated NaHCO₃ and Na₂S₂O₃ solution (1:1; 100 ml each) for 1 h. The organic layer was separated and washed with brine and dried over Na₂SO₄. The solvent was evaporated and a brown oily product was formed.

Yield: 89%

MS (ESI) m/z 257.90 [M+Na]⁺, 290 [M+Na+CH₃OH]⁺

¹H-NMR (CDCl₃, 400 MHz): δ 1.5 (s, 9H), 3.6 (d, J=2.4 Hz, 2H), 7.1 (d, J=8.4 Hz, 2H), 7.35 (d, J=8.4 Hz, 2H), 9.7 (t, J=2.4 Hz, 1H)

### Reaction Procedure H

### Intermediate 28: 2-(prop-2-yn-1-yloxy)ethyl (2-(4-((tert-butoxycarbonyl)amino)phenyl)-1-(diphenoxyphosphoryl)ethyl)carbamate

To a solution of tert-butyl 4-(2-oxoethyl)phenylcarbamate (intermediate 27) (6.99 mmol) and 2-(prop-2-ynyloxy)ethyl carbamate (intermediate 7) (6.99 mmol) in DCM (25 ml), was added triphenyl phosphite (6.99 mmol) and Cu(OTf)₂ (0.699 mmol). The reaction mixture was stirred overnight at room temperature. Solvent was evaporated and the crude mixture dissolved in a small amount of MeOH. Storing the solution at −20° C. yielded a precipitate. Precipitate was filtered off.

Yield: 44%

MS (ESI) m/z 617 [M+Na]⁺

¹H-NMR (CDCl₃, 400 MHz) δ 1.5 (s, 9H), 2.4 (t, J=2.4 Hz, 1H), 2.4-3.55 (m, 1H), 3.3-3.4 (m, 1H), 3.6 (s, 2H), 4.1-4.2 (m, 4H), 4.65-4.77 (m, 1H), 5.03-4.10 (d, J=11.2 Hz, 1H), 6.41 (s, 1H), 7.10-7.36 (m, 14H)

The following intermediates were prepared in a similar way:

### Intermediate 29: 2-(2-(prop-2-yn-1-yloxy)ethoxy)ethyl (2-(4-((tert-butoxycarbonyl)amino)phenyl)-1-(diphenoxyphosphoryl)ethyl)carbamate

Yield: 43%

MS (ESI) m/z 661.4 [M+Na]⁺

¹H-NMR (CDCl₃, 400 MHz) δ 1.5 (s, 9H), 2.4 (t, J=2.4 Hz, 1H), 2.9-3.05 (m, 1H), 3.3-3.4 (m, 1H), 3.55-4.15 (m, 8H), 4.2 (d, J=2.4 Hz, 2H), 4.65-4.75 (m, 1H), 5.10-5.16 (d, J=10.4 Hz, 1H), 6.47 (s, 1H), 7.10-7.35 (m, 14H)

### Intermediate 30: 2-(2-(2-(prop-2-yn-1-yloxy)ethoxy)ethoxy)ethyl (2-(4-((tert-butoxycarbonyl)amino) phenyl)-1-(diphenoxyphosphoryl)ethyl)carbamate

Yield: 53%

MS (ESI) m/z 705.3 [M+Na]⁺

¹H-NMR (CDCl₃, 400 MHz): δ 1.51 (s, 9H), 2.43 (t, J=2.4 Hz, 1H), 2.99 (m, 1H), 3.37 (m, 1H), 3.57-3.71 (m, 10H), 4.12 (m, 2H), 4.19 (d, J=2.4 Hz, 2H), 4.72 (m, 1H), 5.18 (d, J=10.4 Hz, 1H), 6.57 (s, 1H), 7.11-7.35 (m, 14H)

### Intermediate 31: pent-4-yn-1-yl (2-(4-((tert-butoxycarbonyl)amino)phenyl)-1-(diphenoxyphosphoryl)ethyl)carbamate (intermediate 31)

Yield: 49%

MS (ESI) m/z 601.1 [M+Na]⁺

¹H-NMR (CDCl₃, 400 MHz): δ 1.51 (s, 9H), 1.72 (q, J=6.4 Hz, 2H), 1.95 (t, J=2.4 Hz, 1H), 2.15 (t, 2H), 2.97 (m, 1H), 3.35 (m, 1H), 4.06 (t, 2H), 4.71 (m, 1H), 4.98 (d, J=10 Hz, 1H), 6.44 (s, 1H), 7.10-7.35 (m, 14H)

### Intermediate 32: 6-azidohexyl (2-(4-((tert-butoxycarbonyl)amino)phenyl)-1-(diphenoxyphosphoryl)ethyl)carbamate

Yield: 40%

MS (ESI) m/z 660.5 [M+Na]⁺

¹H-NMR (CDCl₃, 400 MHz) δ 1.32 (m, 4H), 1.57 (m, 13H), 2.98 (m, 1H), 3.23 (t, J=6.8 Hz, 2H), 3.35 (m, 1H), 3.97 (m, 2H), 4.73 (m, 1H), 4.99 (d, J=10.4 Hz, 1H), 6.47 (s, 1H), 7.1-7.4 (m, 14H)

### Intermediate 33: 2-(2-azidoethoxy)ethyl (2-(4-(tert-butoxycarbonyl)amino)phenyl)-1-(diphenoxyphosphoryl)ethyl)carbamate

Yield: 51%

MS (ESI) m/z 648.4 [M+Na]⁺

¹H-NMR (CDCl₃, 400 MHz) δ 1.51 (s, 9H), 2.98 (m, 1H), 3.35 (m, 3H), 3.57 (m, 4H), 4.14 (m, 2H), 4.72 (m, 1H), 5.12 (d, J=10 Hz, 1H), 6.46 (s, 1H), 7.10-7.35 (m, 14H)

### Intermediate 34: 2-(2-(2-azidoethoxy)ethoxy)ethyl (2-(4-((tert-butoxycarbonyl)amino)phenyl)-1-(diphenoxyphosphoryl)ethyl)carbamate

Yield: 30%

MS (ESI) m/z 692.5 [M+Na]⁺

¹H-NMR (CDCl₃, 400 MHz) δ 1.51 (s, 9H), 2.98 (m, 1H), 3.35 (m, 3H), 3.63 (m, 8H), 4.14 (m, 2H), 4.72 (m, 1H), 5.13 (d, J=10 Hz, 1H), 6.47 (s, 1H), 7.1-7.4 (m, 14H)

### Intermediate 35: 2-(2-(2-(2-azidoethoxy)ethoxy)ethoxy)ethyl (2-(4-((tert-butoxycarbonyl)amino) phenyl)-1-(diphenoxyphosphoryl)ethyl)carbamate (intermediate 35)

Yield: 22%

MS (ESI) m/z 736.5 [M+Na]⁺

¹H-NMR (CDCl₃ 400 MHz): δ 1.51 (s, 9H), 2.98 (m, 1H), 3.35 (m, 3H), 3.62 (m, 12H), 4.12 (m, 2H), 6.53 (br s, 1H), 7.1-7.4 (m, 14H)

### Reaction Procedure I

### Intermediate 36: pent-4-yn-1-yl (2-(4-((tert-butoxycarbonyl)amino)phenyl)-1-(bis(4-acetamidophenoxy)phosphoryl)ethyl)carbamate

To a solution of tert-butyl 4-(2-oxoethyl)phenylcarbamate (intermediate 26) (5.95 mmol) and 2-(prop-2-ynyloxy)ethyl carbamate (intermediate 11) (5.95 mmol) in THF (50 ml), was added tris(4-acetamidophenyl)phosphite (5.95 mmol) and Cu(OTf)₂ (0.595 mmol). The reaction mixture was stirred overnight at room temperature. Solvent was evaporated and the crude mixture was purified with flash chromatography (100% EtOAc to 10% MeOH in EtOAc)

Yield: 5%

MS m/z 715.3 [M+Na]⁺

¹H-NMR (CDCl₃, 400 MHz) δ 1.51 (s, 9H), 1.97 (br s, 1H), 2.14 (m, 8H), 2.97 (m, 1H), 3.28 (m, 1H), 4.05 (m, 2H), 4.66 (m, 1H), 5.48 (m, 1H), 6.9-7.4 (m, 12H)

### Reaction Procedure J

### Intermediate 37: 2-(prop-2-ynyloxy)ethyl 2-(4-aminophenyl)-1-(diphenoxyphosphoryl)ethyl carbamate 2,2,2-trifluoroacetate

To a solution of 2-(prop-2-yn-1-yloxy)ethyl (2-(4-((tert-butoxycarbonyl)amino)phenyl)-1-(diphenoxyphosphoryl)ethyl)carbamate (intermediate 28) (3.16 mmol) in DMC (25 ml) was added TFA (25 ml). The solution was stirred for 1 hour, followed by evaporation of the solvent. The product was washed with ether.

Yield: 87%

MS (ESI) m/z 495 [M+H]⁺

¹H-NMR (MeOD, 400 MHz) δ 3.83 (s, 1H), 3.03-3.15 (m, 1H), 3.38-3.48 (m, 1H), 3.58-3.64 (s, 2H), 3.97-4.15 (m, 4H), 4.59-4.68 (m, 1H), 7.12-7.51 (m, 14H)

The following intermediates were prepared in a similar way:

### Intermediate 38: 2-(2-(prop-2-ynyloxy)ethoxy)ethyl 2-(4-aminophenyl)-1-(diphenoxyphosphoryl)ethylcarbamate 2,2,2-trifluoroacetate (intermediate 38)

Yield: 93%

MS (ESI) m/z 539 [M+H]⁺

### Intermediate 39: 2-(2-(2-(prop-2-ynyloxy)ethoxy)ethoxy)ethyl 2-(4-aminophenyl)-1-(diphenoxyphosphoryl)ethylcarbamate

Yield: 91%

MS (ESI) m/z 583.2 [M+H]⁺

¹H-NMR (MeOD, 400 MHz): δ 2.87 (t, J=2.4 Hz, 1H), 3.13 (m, 1H), 3.46-3.69 (m, 10H), 3.98-4.15 (m, 2H), 4.18 (d, J=2.4 Hz, 2H), 4.67 (m, 1H), 7.1-8.0 (m, 14H)

### Intermediate 40: pent-4-ynyl 2-(4-aminophenyl)-1-(diphenoxyphosphoryl)ethylcarbamate 2,2,2-trifluoroacetate

Yield: 82%

MS (ESI) m/z 479.1 [M+H]⁺

¹H-NMR (MeOD, 400 MHz): δ 1.7 (q, J=6.4 Hz, 2H), 2.18 (dt, J=2.8 Hz and J=6.8 Hz, 2H), 2.23 (t, J=2.8 Hz, 1H), 3.06 (m, 1H), 3.43 (m, 1H), 4.0 (m, 2H), 4.64 (m, 1H), 7.1-7.5 (m, 14H)

### Intermediate 41: 6-azidohexyl 2-(4-aminophenyl)-1-(diphenoxyphosphoryl)ethylcarbamate 2,2,2-Trifluoroacetate

Yield: 98%

MS (ESI) m/z 538.4 [M+H]⁺

¹H-NMR (MeOD, 400 MHz): δ 1.34 (m, 4H), 1.55 (m, 4H), 3.07 (m, 1H), 3.24 (t, J=6.8 Hz, 2H), 3.41 (m, 1H), 3.86 (m, 1H), 3.99 (m, 1H), 4.64 (m, 1H), 7.10-7.50 (m, 14H)

### Intermediate 42: 2-(2-azidoethoxy)ethyl 2-(4-aminophenyl)-1-(diphenoxyphosphoryl)ethyl carbamate 2,2,2-trifluoroacetate (intermediate 42)

Yield: 98%

MS (ESI) m/z 526.3 [M+H]⁺

¹H-NMR (MeOD, 400 MHz) δ 3.10 (m, 1H), 3.44 (m, 1H), 3.60 (m, 4H), 4.02 (m, 1H), 4.10 (m, 1H), 4.66 (m, 1H), 7.15-7.55 (m, 14H) One CH₂ from the azidoethoxy side chain is hidden behind the MeOH peak

### Intermediate 43: 2-(2-(2-azidoethoxy)ethoxy)ethyl 2-(4-aminophenyl)-1-(diphenoxyphosphoryl)ethyl carbamate 2,2,2-trifluoroacetate

Yield: 97%

MS (ESI) m/z 570.4 [M+H]⁺

¹H-NMR (MeOD, 400 MHz) δ 3.09 (m, 1H), 3.44 (m, 1H), 3.61 (m, 8H), 3.99 (m, 1H), 4.11 (m, 1H), 4.64 (m, 1H), 7.0-7.54 (m, 14), one CH₂ is hidden by the MeOH peak

### Intermediate 44: 2-(2-(2-(2-azidoethoxy)ethoxy)ethoxy)ethyl 2-(4-aminophenyl)-1-(diphenoxyphosphoryl)ethylcarbamate 2,2,2-trifluoroacetate

Yield: 98%

MS (ESI) m/z 614.4 [M+H]⁺, 636.4 [M+Na]⁺

### Intermediate 45: pent-4-ynyl 2-(4-aminophenyl)-1-(bis(4-acetamidophenoxy)phosphoryl)ethyl carbamate 2,2,2-trifluoroacetate

Yield: 82%

MS (ESI) m/z 593.2 [M+H]⁺

¹H-NMR (MeOD, 400 MHz) δ 1.70 (m, 2H), 2.11 (s, 6H), 2.17 (m, 2H), 2.25 (t, J=2.4 Hz, 1H), 3.04 (m, 1H), 3.41 (m, 1H), 3.96 (m, 2H), 4.59 (m, 1H), 7.11 (m, 4H), 7.18 (d, J=8.4 Hz, 2H), 7.38 (d, J=8.4 Hz, 2H), 7.54 (d, J=8.8 Hz, 4H)

### Intermediate 55: 2-(prop-2-ynyloxy)ethyl 1-(diphenoxyphosphoryl)-2-(4-guanidinophenyl)ethyl carbamate 2,2,2-trifluoroacetate

Yield: 95%

MS (ESI) m/z 537 [M+H]⁺

¹H NMR (MeOD, 400 MHz) δ 2.82 (t, J=2.4 Hz, 1H), 3.03-3.14 (m, 1H), 3.37-3.44 (m, 1H), 3.61-3.65 (m, 2H), 4.03-4.16 (m, 4H), 4.58-4.67 (m, 1H), 7.15-7.45 (m, 14H)

LC-MS tᵣ 14.3 min (97.4%)

HPLC (214 nm) tᵣ 18.23 min (100%)

HPLC (254 nm) tᵣ 18.23 min (100%)

### Intermediate 56: 2-(2-(prop-2-ynyloxy)ethoxy)ethyl 1-(diphenoxyphosphoryl)-2-(4-guanidino phenyl)ethylcarbamate 2,2,2-trifluoroacetate

Yield: 98%

MS (ESI) m/z 581 [M+H]⁺

¹H NMR (MeOD, 400 MHz) δ 2.84 (t, J=2.4 Hz, 1H), 3.03-3.15 (m, 1H), 3.36-3.45 (m, 1H), 3.53-4.18 (m, 10H), 4.57-4.69 (m, 1H), 7.16-7.99 (m, 14H)

LC-MS tᵣ 13.8 min (97.8%)

HPLC (214 nm) tᵣ 18.29 min (100%)

HPLC (254 nm) tᵣ 18.32 min (100%)

### Intermediate 57: 2-(2-(2-(prop-2-ynyloxy)ethoxy)ethoxy)ethyl 1-(diphenoxyphosphoryl)-2-(4-guanidinophenyl)ethylcarbamate 2,2,2-trifluoroacetate

Yield: 75%

MS (ESI) m/z 625 [M+H]⁺

¹H NMR (MeOD, 400 MHz) δ 2.82 (t, 1H, J=J′=2.4), 3.03-3.15 (m, 1H), 3.37-3.45 (m, 1H), 3.54-4.19 (m, 14H), 4.58-4.70 (m, 1H), 7.16-7.97 (m, 14H)

LC-MS tᵣ 14.0 min (96.5%)

HPLC (214 nm) tᵣ 18.43 min (100%)

HPLC (254 nm) tᵣ 18.42 min (100%)

### Intermediate 58: pent-4-ynyl 1-(diphenoxyphosphoryl)-2-(4-guanidinophenyl)ethylcarbamate 2,2,2-trifluoroacetate

Yield: 61%

MS (ESI) m/z 521.2 [M+H]⁺

¹H-NMR (MeOD, 400 MHz): δ 1.72 (q, J=6.8 Hz, 2H), 2.20 (dt, J=7.2 Hz and 2.4 Hz, 2H), 2.23 (t, J=2.4 Hz, 1H), 3.07 (m, 1H), 3.40 (m, 1H), 4.03 (m, 2H), 4.64 (m, 1H), 7.15-7.45 (m, 14H)

LC-MS tᵣ 14.3 min (99%)

HPLC (UV 214 nm) tᵣ 19.07 min (99.1%)

HPLC (UV 254 nm) tᵣ 19.20 min (100%)

### Intermediate 59: 6-azidohexyl 1-(diphenoxyphosphoryl)-2-(4-guanidinophenyl)ethylcarbamate 2,2,2-trifluoroacetate

Yield: 75%

MS (ESI) m/z 580.3 [M+H]⁺

¹H-NMR (MeOD, 400 MHz): δ 1.35 (m, 4H), 1.56 (m, 4H), 3.15 (m, 1H), 3.25 (m, 3H), 3.43 (m, 1H), 3.98 (m, 2H), 4.65 (m, 1H), 7.18-7.94 (m, 14H)

LC-MS tᵣ 11.2 min (100%)

### Intermediate 60: 2-(2-azidoethoxy)ethyl 1-(diphenoxyphosphoryl)-2-(4-guanidinophenyl)ethyl carbamate 2,2,2-trifluoroacetate

Yield: 45%

MS (ESI) m/z 568.4 [M+H]⁺

¹H-NMR (MeOD, 400 MHz) δ 3.10 (m, 1H), 3.41 (m, 1H), 3.60 (m, 4H), 4.08 (m, 2H), 4.63 (m, 1H), 7.15-7.45 (m, 14H)

LC-MS tᵣ 14.7 min (99.1%)

HPLC (214 nm) tᵣ 19.01 min(97%)

HPLC (254 nm) tᵣ 18.92 min (97%)

### Intermediate 61: 2-(2-(2-azidoethoxy)ethoxy)ethyl 1-(diphenoxyphosphoryl)-2-(4-guanidinophenyl)ethylcarbamate 2,2,2-trifluoroacetate

Yield: 63%

MS (ESI) m/z 612.4 [M+H]⁺

¹H-NMR (MeOD, 400 MHz): δ 3.1 (m, 1H), 3.31 (m, 2H), 3.42 (m, 1H), 3.62 (m, 8H), 4.04 (m, 1H), 4.11 (m, 1H), 4.63 (m, 1H), 7.15-7.45 (m, 14H)

LC-MS tᵣ 14.6 min (93.3%)

HPLC (214 nm) tᵣ 18.9 min (91.1%)

### Intermediate 62: 2-(2-(2-(2-azidoethoxy)ethoxy)ethoxy)ethyl 1-(diphenoxyphosphoryl)-2-(4-guanidinophenyl)ethylcarbamate 2,2,2-trifluoroacetate

Yield: 78%

MS (ESI) m/z 656.5 [M+H]⁺

HPLC (214 nm) tᵣ 18.8 min (95.5%)

¹H-NMR (MeOD, 400 MHz): δ 3.09 (m, 1H), 3.35 (t, J=5.2 Hz, 2H), 3.40 (m, 1H), 3.63 (m, 12H), 4.05 (m, 1H), 4.11 (m, 1H), 4.64 (m, 1H), 7.15-7.45 (m, 14H)

### Intermediate 63: pent-4-ynyl 1-(bis(4-acetamidophenoxy)phosphoryl)-2-(4-guanidinophenyl)ethyl carbamate 2,2,2-trifluoroacetate

Yield: 67%

MS (ESI) m/z 635.3 [M+H]⁺

¹H-NMR (MeOD, 400 MHz) δ 1.71 (m, 2H), 2.11 (s, 6H), 2.18 (m, 2H), 2.23 (t, J=2.8 Hz, 1H), 3.12 (m, 1H), 3.40 (m, 1H), 4.02 (m, 1H), 7.13 (m, 4H), 7.22 (d, J=8.4 Hz, 2H), 7.40 (d, J=8.4 Hz, 2H), 7.55 (m, 4H)

LC-MS tᵣ 11.4 (96.5%)

### Reaction Procedure K

### Intermediate 46: 2-(prop-2-yn-1-yloxy)ethyl (1-(diphenoxyphosphoryl)-2-(4-((2,3-bis-(tert-butoxycarbonyl)guanidine)phenyl)ethyl)carbamate

To a solution of 2-(prop-2-ynyloxy)ethyl 2-(4-aminophenyl)-1-(diphenoxyphosphoryl)ethyl carbamate 2,2,2-trifluoroacetate (intermediate 37) (1.890 mmol) in CHCl₃ (50 ml) was added (Z)-tert-butyl (1H-pyrazol-1-yl)methanediylidenedicarbamate (1.890 mmol) and triethylamine (5.67 mmol). The solution was allowed to stir for 3 days at room temperature. Solvent was evaporated and the crude mixture was dissolved in EtOAc and washed with 1N HCl, saturated NaHCO₃ and brine solution. The organic solvent was dried over anhydrous Na₂SO₄, filtered, evaporated and purified by flash chromatography (100% hexane to 30% EtOAc in hexane).

Yield: 32%

MS (ESI) m/z 737 [M+1]⁺

¹H-NMR (CDCl₃, 400 MHz) δ 1.48 (s, 9H), 1.53 (s, 9H), 2.44 (s, 1H), 2.93-3.07 (m, 1H), 3.32-3.43 (m, 1H), 3.63 (d, J=4.4 Hz, 2H), 4.12-4.23 (m, 4H), 4.66-4.81 (m, 1H), 5.05 (d, J=10 Hz, 1H), 7.10-7.35 (m, 14H), 7.57 (d, J=8.4 Hz, 2H), 10.3 (s, 1H), 11.6 (s, 1H)

The following intermediates were prepared in a similar way:

### Intermediate 47: 2-(2-(prop-2-yn-1-yloxy)ethoxy)ethyl (1-(diphenoxyphosphoryl)-2-(4-(2,3-bis-(tert-butoxycarbonyl)guanidine)phenyl)ethyl)carbamate

Yield: 60%

MS (ESI) m/z 781 [M+H]⁺, 803 [M+Na]⁺, 819 [M+K]⁺

¹H-NMR (CDCl₃, 400 MHz) δ 1.51 (s, 9H), 1.54 (s, 9H), 2.42 (t, J=2.4 Hz, 1H), 2.94-3.07 (m, 1H), 3.33-3.43 (m, 1H), 3.55-4.20 (m, 10H), 4.68-4.81 (m, 1H), 5.06 (d, J=9.6 Hz, 1H) 7.10-7.59 (m, 14H), 10.30 (s, 1H), 11.60 (s, 1H)

### Intermediate 48: 2-(2-(2-(prop-2-yn-1-yloxy)ethoxy)ethoxy)ethyl (1-(diphenoxyphosphoryl)-2-(4-(2,3-bis(tert-butoxycarbonyl)guanidino)phenyl)ethyl)carbamate

Yield: 76%

MS (ESI) m/z 825 [M+H]⁺, 847 [M+Na]⁺

¹H-NMR (CDCl₃,400 MHz) δ 1.48 (s, 9H), 1.53 (s, 9H), 2.41 (t, 1H), 2.96-3.07 (m, 1H), 3.33-3.42 (m, 1H), 3.65-4.20 (m, 12H), 4.68-4.81 (m, 1H), 5.1-5.16 (d, J=10 Hz, 1H), 7.11-7.35 (m, 14H), 7.53-7.58 (d, J=8.4 Hz, 2H)

### Intermediate 49: pent-4-yn-1-yl (1-(diphenoxyphosphoryl)-2-(4-(2,3-bis(tert-butoxycarbonyl) guanidine)phenyl)ethyl)carbamate

Yield: 61%

MS (ESI) m/z 743.1 [M+Na]⁺

¹H-NMR (CDCl₃, 400 MHz): δ 1.51 (d, 18H), 1.73 (q, J=6.4 Hz, 2H), 1.96 (t, J=2.8 Hz, 1H), 2.17 (t, 2H), 3.01 (m, 1H), 3.38 (m, 1H), 4.08 (t, J=6 Hz, 2H), 4.76 (m, 1H), 4.96 (d, J=10.4 Hz, 1H), 7.0-7.6 (m, 14H), 10.33 (s, 1H), 11.63 (s, 1H)

### Intermediate 50: 6-azidohexyl (1-(diphenoxyphosphoryl)-2-(4-(2,3-bis(tert-butoxycarbonyl)guanidine)phenyl)ethyl)carbamate

Yield: 35%

MS (ESI) m/z 780.6 [M+H]⁺

¹H-NMR (CDCl₃, 400 MHz): δ 1.32 (m, 4H), 1.52 (m, 22H), 3.02 (m, 1H), 3.22 (t, J=6.8 Hz, 2H), 3.37 (m, 1H), 3.97 (t, J=6.4 Hz, 2H), 4.77 (m, 1H), 4.93 (d, J=10.8 Hz, 1H), 7.1-7.6 (m, 14H), 10.31 (s, 1H), 11.61 (s, 1H)

### Intermediate 51: 2-(2-azidoethoxy)ethyl (1-(diphenoxyphosphoryl)-2-(4-(2,3-bis(tert-butoxycarbonyl)guanidine)phenyl)ethyl)carbamate

Yield: 36%

MS (ESI) m/z 768.5 [M+H]⁺, 790.6 [M+Na]⁺

¹H-NMR (CDCl₃, 400 MHz): δ1.53 (d, 18H), 3.01 (m, 1H), 3.32 (t, J=4.8 Hz, 2H), 3.40 (m, 1H), 3.59 (m, 4H), 4.15 (m, 2H), 4.75 (m, 1H), 5.09 (d, J=10.4 Hz, 1H), 7.10-7.60 (m, 14H), 10.31 (s, 1H), 11.6 (s, 1H)

### Intermediate 52: 2-(2-(2-azidoethoxy)ethoxy)ethyl (1-(diphenoxyphosphoryl)-2-(4-(2,3-bis(tert-butoxycarbonyl)guanidino)phenyl)ethyl)carbamate

Yield: 37%

MS (ESI) m/z 812.5 [M+H]⁺, 834.5 [M+Na]⁺

¹H-NMR (CDCl₃, 400 MHz): δ 1.53 (d, 18H), 3.02 (m, 1H), 3.38 (m, 3H), 3.63 (m, 8H), 4.13 (m, 2H), 4.75 (m, 1H), 5.13 (s, J=10 Hz, 1H), 7.1-7.6 (m, 14H), 10.3 (s, 1H), 11.6 (s, 1H)

### Intermediate 53: 2-(2-(2-(2-azidoethoxy)ethoxy)ethoxy)ethyl (1-(diphenoxyphosphoryl)-2-(4-(2,3-bis(tert-butoxycarbonyl)guanidino)phenyl)ethyl)carbamate

Yield: 27%

MS (ESI) m/z 878.6 [M+Na]⁺

¹H-NMR (CDCl₃, 400 MHz): δ 1.52 (d, 18H), 3.02 (m, 1H), 3.39 (m, 3H), 3.63 (m, 12H), 4.14 (m, 2H), 4.75 (m, 1H), 5.15 (d, 1H), 7.1-7.6 (m, 14H), 10.3 (s, 1H), 11.6 (s, 1H)

### Reaction Procedure L

### Intermediate 54: pent-4-yn-1-yl (1-(bis(4-acetamidophenoxy)phosphoryl)-2-(4-(2,3-bis(tert-butoxycrabonyl)guanidine)phenyl)ethyl)carbamate

To a solution of pent-4-ynyl 2-(4-aminophenyl)-1-(bis(4-acetamidophenoxy)phosphosphoryl)ethylcarbamate 2,2,2-trifluoroacetate (intermediate 45) (0.283 mmol) in DCM (3 ml) and Acetonitrile (Volume: 3.00 ml) was added, Boc-pyrazolguanidine (0.566 mmol) and TEA (0.849 mmol). The solution was allowed to stir for 5 hours. Extra Boc-pyrazolguanidine (0.566 mmol) was added and the solution was allowed to stir for another 48 hours. Solvent was evaporated and the crude product dissolved in EtOAc. The organic layer was washed with 2N HCl, sat NaHCO₃ and brine solution. It was dried over Na₂SO₄, filtered and evaporated. Crude product was purified using 15 flash chromatography (2.5% MeOH in EtOAc).

Yield: 34%

MS (ESI) m/z 857.3 [M+Na]⁺

¹H-NMR (CDCl₃, 400 MHz) δ 1.46 (s, 9H), 1.54 (s, 9H), 1.72 (m, 2H), 2.0 (m, 7H), 2.17 (m, 2H), 2.89 (m, 1H), 3.20 (m, 1H), 4.08 (m, 2H), 4.64 (m, 1H), 6.38 (d, J=10 Hz, 1H), 6.8-7.5 (m, 12H), 8.46 (s, 1H), 8.64 (s, 1H), 10.19 (s, 1H), 11.62 (s, 1H)

### Reaction Procedure M

### Intermediate 65: 8-azidooctanoic acid

To a stirred solution of 8-bromooctanoic acid (intermediate 64) (2.241 mmol) in DMF (5 ml), was added sodium azide (4.48 mmol), and the mixture was heated at 85° C. for 3 h. The crude reaction mixture was diluted in DCM (50 ml) and this solution was washed with 0.1 N HCl. The organic layer was dried over anhydrous Na₂SO₄. The solvent was evaporated. A yellow oily product was obtained.

Yield: 79%

MS (ESI) m/z 184.0 [M−H]⁻

¹H-NMR (CDCl₃, 400 MHz): δ 1.37 (m, 6H), 1.63 (m, 4H), 2.35 (t, J=7.2 Hz, 2H), 3.25 (t, J=6.8 Hz, 2H)

### Reaction Procedure N

### Intermediate 67: N-(6-(diethylamino)-9-(2-(piperazine-1-carbonyl)phenyl)-3H-xanthen-3-ylidene)-Nethylethanaminium chloride

A 1.0 M solution of trimethylaluminium (4.52 mmol) in heptane was added dropwise to a solution of piperazine (9.04 mmol) in 3.5 ml CH₂Cl₂ at room temperature. Gas evolution was observed during the addition period. After 1 h stirring a white precipitate was observed. A solution of rhodamine B base (intermediate 66) (2.26 mmol) in 2 ml CH₂Cl₂ was added dropwise to the heterogeneous solution. The solution was refluxed for 48 h (65° C.). A 0.1 M aqueous solution of HCl was added dropwise until gas evolution ceased. The heterogeneous solution was filtered and the retained solids were rinsed with CH₂Cl₂ and a 4:1 CH₂Cl₂/MeOH solution. The combined filtrate was concentrated and the residue was dissolved in CH₂Cl₂, filtered to remove insoluble salts and concentrated again. The resulting glassy solid was then partitioned between dilute aqueous NaHCO₃ (2% m/v) and EtOAc. After isolation, the aqueous layer was washed with 3 additional portions of EtOAc to remove residual starting material. The retained aqueous layer was saturated with NaCl, acidified with 1 M aqueous HCl, and then extracted with multiple portions of 2:1 iPrOH/CH₂Cl₂, until a faint pink color persisted. The combined organic layers were then dried over Na₂SO₄, filtered and concentrated under reduced pressure. The glassy purple solid was dissolved in a minimal amount of MeOH and precipitated by dropwise addition to a large volume of Et₂O. The product was collected by filtration as a dark purple solid. Further purification was performed using flash chromatography (100% DMC to 15% MeOH in DCM).

Yield: 15%

MS (ESI) m/z 511.80 [M]⁺

¹H-NMR (MeOD, 400 MHz): δ 1.32 (t, J=7.2 Hz, 12H), 2.70 (br s, 4H), 3.41 (br s, 4H), 3.70 (q, J=7.2 Hz, 8H), 6.97 (d, J=2.4 Hz, 2H), 7.08 (dd, J=2.8 Hz and J=9.2 Hz, 2H), 7.27 (d, J=9.6 Hz, 2H), 7.51 (m, 1H), 7.68 (m, 1H), 7.70 (m, 2H)

HPLC (214 nm) tᵣ 18.08 min (100%)

### Reaction Procedure O

### Intermediate 68: N-(9-(2-(4-(8-azidooctanoyl)piperazine-1-carbonyl)phenyl)-6-(diethylamino)-3H-xanthen-3-ylidene)-N-ethylethanaminium chloride

To a solution of 8-azidooctanoic acid (intermediate 65) (1.828 mmol) in DMF (20 ml) was added TBTU (2.010 mmol) and DIPEA (5.48 mmol). The solution was stirred for 15 min at RT. N-(6-(diethylamino)-9-(2-(piperazine-1-carbonyl)phenyl)-3H-xanthen-3-ylidene)-N-ethylethanaminium chloride (intermediate 67) (1.828 mmol) was added and the solution was allowed to stir overnight at RT. 200 ml water was added and the aqueous layer was extracted with DCM (2×). The resulting organic layer was extracted with 2N HCl, Saturated NaHCO₃ and Brine solution. The solvent was dried over Na₂SO₄, filtered and evaporated. The formed product was washed with hexane, EtOAc/Hexane % and 2×20 ml EtOAc/Hexane 1/1. A dark purple solid was formed. Product was dissolved in a small amount of DCM and added to a large amount of ether. A precipitate was formed.

Yield: 87%

MS (ESI) m/z 678.7 [M]⁺

¹H-NMR (MeOD, 400 MHz): δ 1.31 (m, 18H), 1.56 (m, 4H), 2.34 (t, J=7.6 Hz, 2H), 3.27 (t, J=6.4 Hz, 2H), 3.35 (br s, 8H), 3.70 (q, J=7.2 Hz, 8H), 6.96 (d, J=2.4 Hz, 2H), 7.07 (dd, J=2.4 Hz and J=9.6 Hz, 2H), 7.28 (d, J=9.6 Hz, 2H), 7.52 (m, 1H), 7.70 (m, 1H), 7.77 (m, 2H)

LC-MS tᵣ 21.5 min (100%)

HPLC (214 nm) tᵣ 27.3 min (100%)

HPLC (254 nm) tᵣ 27.4 min (100%)

### Reaction Procedure P

### Intermediate 69: 8-azidooctanoyl chloride

To a stirred solution of 8-azidooctanoic acid (intermediate 65) (2 g, 10.80 mmol) in anhydrous toluene (Volume: 50 ml), was added oxalylchloride (16.20 mmol). A catalytic amount of DMF (2 drops) was added and the solution was stirred at room temperature for 4 h. A white precipate was observed. After concentration in vacuo, the residue was co-evaporated with toluene. A red product was obtained and was used in the next step without further purification.

### Reaction Procedure Q

### Intermediate 71: 2,5-dioxopyrrolidin-1-yl 5-((3aS,4S,6aR)-2-oxohexahydro-1H-thieno[3,4-d]imidazol-4-yl)pentanoate

D-Biotin (0.819 mmol) and N-hydroxysuccinimide (0.819 mmol) were dissolved into 6 ml hot anhydrous DMF (70° C.) in a 50 ml round-bottom flasks with stirring. DCC (1.064 mmol) was added and the solution was stirred overnight at room temperature. The formed DCU was filtered off and the solution was evaporated to dryness. The residue was taken up into boiling isopropanol and the solution was allowed to cool down to RT. The target compound was precipitated out and the product was filtered off.

Yield: 79%

MS (ESI) m/z 364.1 [M+Na]⁺

¹H-NMR (DMSO, 400 MHz): δ 1.42-1.67 (m, 6H), 2.57-2.60 (d, J=12.4 Hz, 1H), 2.68 (t, J=7.3 Hz, 2H), 2.81-2.90 (m, 5H), 3.11 (m, 1H), 4.14-4.17 (m, 1H), 4.29-4.35 (m, 1H), 6.37 (s, 1H), 6.42 (s, 1H)

### Reaction Procedure R

### Intermediate 72: N-(2-(2-(2-(2-azidoethoxy)ethoxy)ethoxy)ethyl)-5-((3aS,4S,6aR)-2-oxohexahydro-1H-thieno[3,4-d]imidazol-4-yl)pentanamide

TEA (0.879 mmol) was added to a solution of 2-(2-(2-(2-azidoethoxy)ethoxy)ethoxy)ethanamine (0.879 mmol) in 15 ml DMF, followed by the addition of biotin-NHS (intermediate 71) (0.879 mmol) in 10 ml DMF. The resulting solution was stirred at room temperature for 15 hours. The solvent evaporated and the obtained crude mixture was purified via flash chromatography (100% EtOAc to 20% MeOH in EtOAc)

Yield: 77%

MS (ESI) m/z 467.3 [M+Na]⁺

¹H-NMR (DMSO, 400 MHz): δ 1.29-1.64 (m, 6H), 2.07 (t, J=7.2 Hz, 2H), 2.59 (d, J=12.8 Hz, 1H), 2.82 (dd, J=5.2 Hz and J=12.4 Hz, 1H), 3.09 (m, 1H), 3.18 (m, 2H), 3.38 (m, 4H), 3.53 (m, 8H), 3.62 (m, 2H), 4.12 (m, 1H), 4.31 (m, 1H), 6.36 (s, 1H), 6.42 (s, 1H), 7.83 (t, J=5.6 Hz, 1H)

### Reaction Procedure S

### Intermediate 73: 10-(7-azidoheptyl)-5,5-difluoro-1,3,7,9-tetramethyl-5H-dipyrrolo[1,2-c:2′,1′-f][1,3,2]diazaborinin-4-ium-5-uide

A 1M solution of 8-azidooctanoyl chloride (Intermediate 66) (10.80 mmol) in 10 ml DCE was made up and 2,4-dimethyl-pyrrole (22.68 mmol)was added. The resulting mixture was stirred at 65° C. for 2 hours. After leaving to cool to room temperature. Borontrifluorideethercomplex (54.0 mmol) was added dropwise, followed by the dropwise addition of N,N-Di-iso-propylethylamine (43.2 mmol). N₂ gas was then bubbled through the solution, and the reaction mixture was stirred overnight at ambient temperature. H₂O and EtOAc was added. The organic layer was dried over Na₂SO₄ and the crude product was obtained after evaporation of the solvent. Purification via flash chromatography (7% EtOAc in Hexane) yielded the desired product.

Yield: 6%

MS (ESI) m/z 410.1 [M+Na]⁺

¹H-NMR (CDCl₃, 400 MHz): δ 1.30-1.7 (m, 10H), 2.414 (s, 6H), 2.514 (s, 6H), 2.94 (m, 2H), 3.26 (m, 2H), 6.052 (s, 2H)

### Reaction Procedure T

### Intermediate 75: N-(2-(2-(2-(2-azidoethoxy)ethoxy)ethoxy)ethyl)-4-fluorobenzamide

2-(2-(2-(2-azidoethoxy)ethoxy)ethoxy)ethanamine (intermediate 74) (2 mmol) was dissolved in 2 ml DCM and TEA (3.00 mmol). 2,5-dioxopyrrolidin-1-yl 4-fluorobenzoate (2.200 mmol) dissolved in 1 ml DCM was added to the solution. The resulting mixture was stirred for 4 h at room temperature. The crude reaction was then diluted with DCM and washed with 10% citric acid solution and brine. The organic layer was dried over Na₂SO₄, filtered, and concentrated under reduced pressure. Flash chromatography (100% DCM to 4% MeOH in DCM) yielded the desired product.

Yield: 74%

¹H-NMR (CDCl₃, 400 MHz): δ 3.27 (m, 2H), 3.57 (m, 14H), 6.90 (br s, 1H), 7.02 (m, 2H), 7.76 (m, 2H)

### Reaction Procedure U

### Intermediate 77: 2-((1E,3E,5E)-5-(1-(6-((2-azidoethyl)amino)-6-oxohexyl)-3,3-dimethylindolin-2-ylidene)penta-1,3-dien-1-yl)-1,3,3-trimethyl-3H-indol-1-ium

To a solution of 2-((1E,3E,5E)-5-(1-(5-carboxypentyl)-3,3-dimethylindolin-2-ylidene)penta-1,3-dien-1-yl)-1,3,3-trimethyl-3H-indol-1-ium (intermediate 76) (0.193 mmol) in 4 ml DMF was added TBTU (0.231 mmol) and DIPEA (0.424 mmol). The resulting solution was allowed to stir for 1 hour at room temperature before 2-azidoethylamine (0.267 mmol) was added. This final solution was stirred overnight at room temperature. DMF was removed under reduced pressure, followed by flash chromatography (100% DCM to 10% MeOH in DCM)

Yield: 57%

¹H-NMR (MeOD, 400 MHz): δ 1.46 (m, 2H), 1.68 (m, 14H), 1.75 (m, 2H), 2.22 (t, J=7.2 Hz, 2H), 3.30 (s, 4H), 3.61 (s, 3H), 4.09 (t, J=7.6 Hz, 2H), 6.25 (dd, J=2.8 Hz and J=13.6 Hz, 2H), 6.62 (t, J=12.4 Hz, 1H), 7.26 (m, 4H), 7.39 (m, 2H), 7.47 (d, J=7.6 Hz, 2H), 8.22 (t, J=13.6 Hz, 2H)

LC-MS tᵣ 19.0 min (98.7%)

2-azidoethylamine was synthesized according to a procedure from following reference: Benalil, A.; Carboni, B.; Vaultier, M. Synthesis of 1,2-aminoazides. Conversion to unsymmetrical vicinal diamines by catalytic hydrogenation or reductive alkylation with dichloroboranes. Tetrahedron 1991, 47, 8177-8194

### Synthesis of Final Compounds:

For synthesizing the final compounds either the Boc protected guanidine intermediates (intermediates 46-54) or the unprotected guanidine intermediates (intermediates 55-63) can be used. By using click-chemistry (Cu(I) catalyzed Huisgen azide alkyne cycloaddition), different visualization tags (e.g. rhodamine, biotin, BODIPY) can be attached at the intermediates.

In case of the rhodamine and BODIPY final products, the “click” reaction was performed on the unprotected guanidine intermediates, whereas biotin and 4-fluorenzamide were coupled on the boc-protected guanidine intermediates with a deprotection as final step.

Rhodamine-azide (intermediate 68) was coupled with the alkyne-probes (intermediates 55-58, 63) and it was noticed that the final compound which possesses the linker part of intermediate 58/63, has the fastest inhibition rate constant. Therefore further couplings of different visualization tags were only performed on intermediate 58 or on its protected analog (intermediate 49).

The “click”-reaction was performed at room temperature in aqueous media in the presence of in situ generated Cu(I) catalyst (CuSO₄/Na Ascorbate). (Scheme 4 and 5)

In a similar way it was possible to synthesize a uPA activity based probe with a Cy5 type visualization linker.

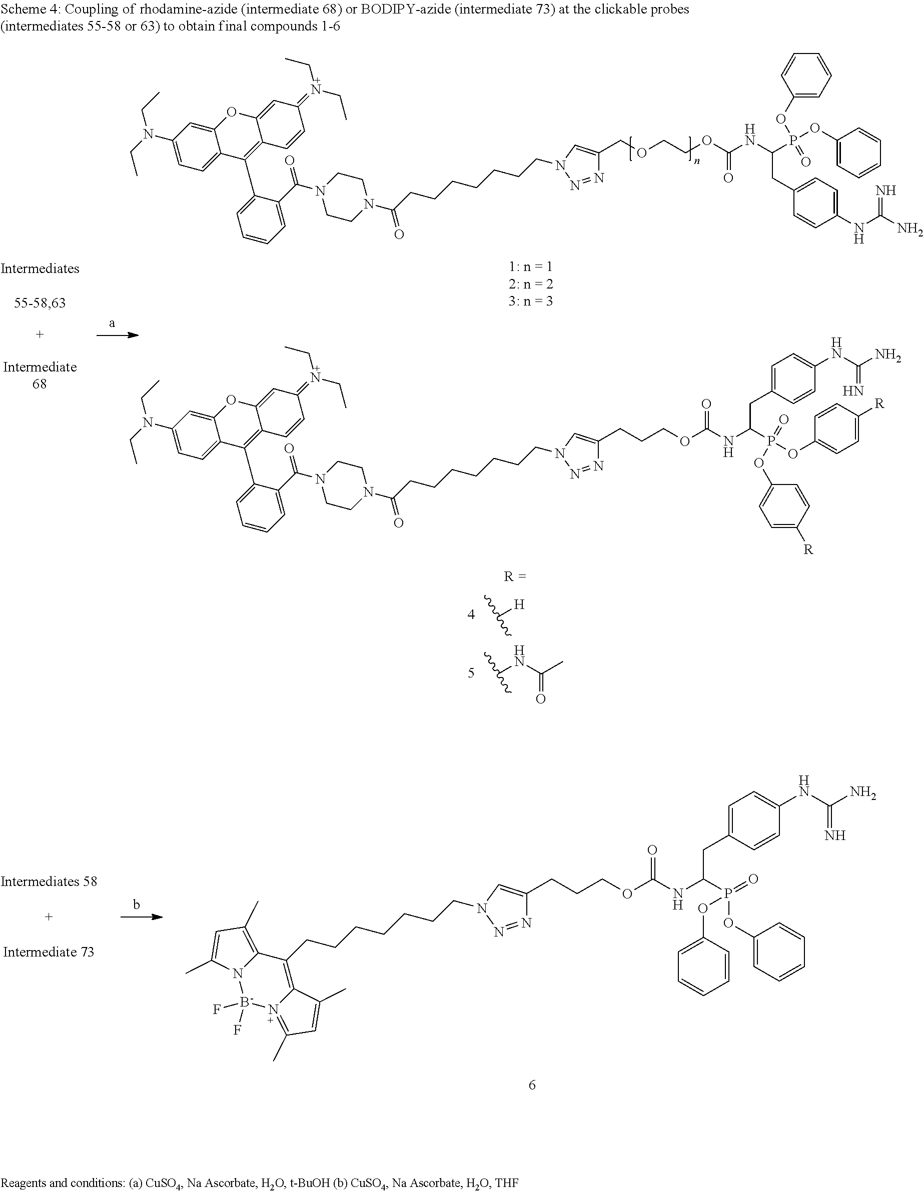

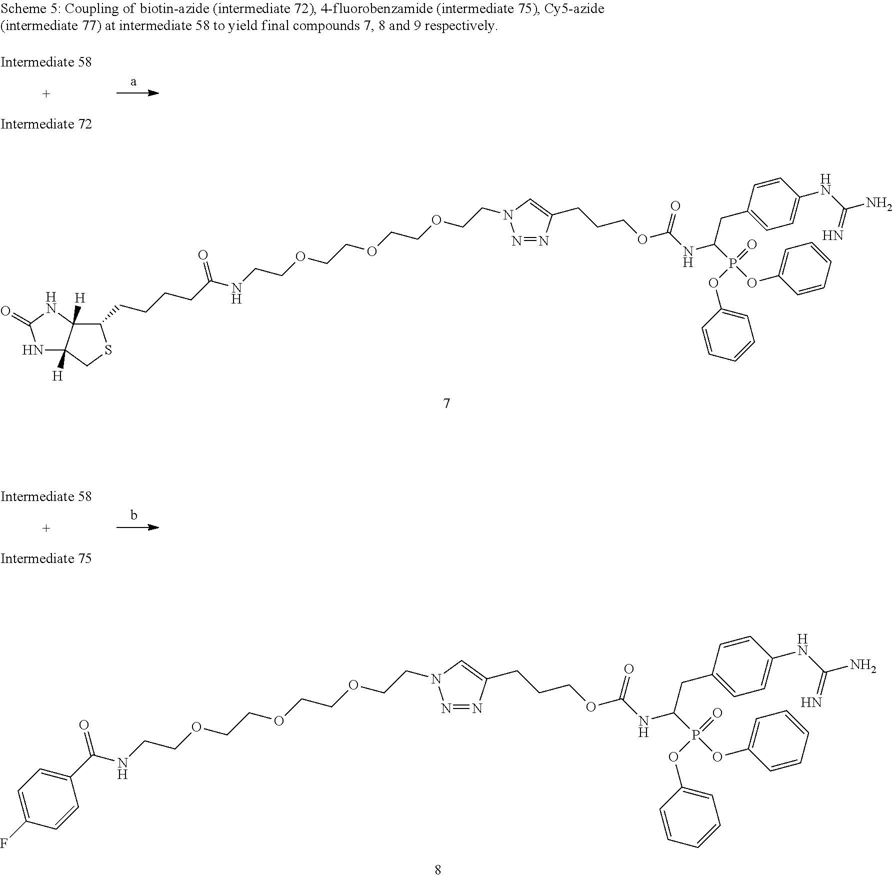 [SMILES: N=C(N)Nc1ccc(CC(NC(=O)OCCCc2cn(CCOCCOCCOCCNC(=O)CCCC[C@@H]3SC[C@@H]4NC(=O)N[C@@H]43)nn2)P(=O)(Oc2ccccc2)Oc2ccccc2)cc1 ; N=C(N)Nc1ccc(CC(NC(=O)OCCCc2cn(CCOCCOCCOCCNC(=O)c3ccc(F)cc3)nn2)P(=O)(Oc2ccccc2)Oc2ccccc2)cc1]

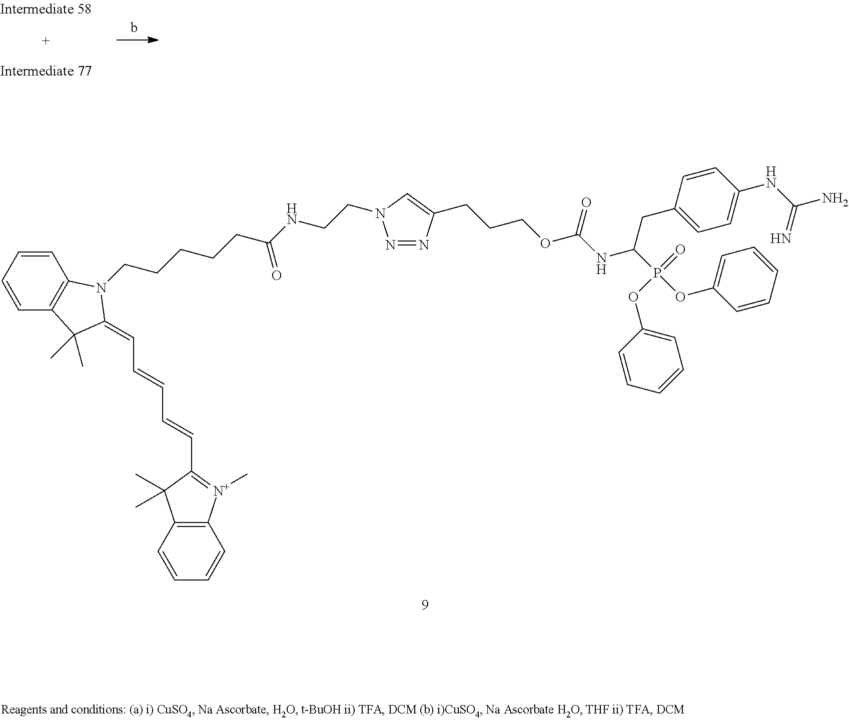 [SMILES: C[N+]1=C(/C=C/C=C/C=C2/N(CCCCCC(=O)NCCn3cc(CCCOC(=O)NC(Cc4ccc(NC(=N)N)cc4)P(=O)(Oc4ccccc4)Oc4ccccc4)nn3)c3ccccc3C2(C)C)C(C)(C)c2ccccc21]

Biochemical Evaluation.

IC₅₀ values of intermediates 55-63 and final compounds 1-9 were determined for uPA and for other representative trypsin-like serine proteases. The latter are involved in the blood coagulation cascade and fibrinolysis: tPA, plasmin, thrombin, and FXa.

### Synthesis of Final Compounds

### Reaction Procedure V

Final compound 1: 2,2,2-trifluoroacetic acid, N-(6-(diethylamino)-9-(2-(4-(8-(4-((2-(((1-(diphenoxyphosphoryl)-2-(4-guanidinophenyl)ethyl)carbamoyl)oxy)ethoxy)methyl)-1H-1,2,3-triazol-1-yl)octanoyl)piperazine-1-carbonyl)phenyl)-3H-xanthen-3-ylidene)-N-ethylethanaminium chloride

2-(prop-2-ynyloxy)ethyl 1-(diphenoxyphosphoryl)-2-(4-guanidinophenyl)ethylcarbamate 2,2,2-trifluoroacetate (intermediate 55) (0.154 mmol), N-(9-(2-(4-(8-azidooctanoyl)piperazine-1-carbonyl)phenyl)-6-(diethylamino)-3H-xanthen-3-ylidene)-N-ethylethanaminium chloride (Intermediate 68) (0.154 mmol), sodium ascorbate (0.369 mmol) and copper(II) sulfate (0.092 mmol) were dissolved in water (400 μl) and t-BuOH (400 μl). The solution was allowed to stir for 5 hours at room temperature. DCM (40 ml) and H₂O (40 ml) were added. Organic layer is separated and washed with brine solution, dried over anhydrous Na₂SO₄, filtered and removed in vacuo. Crude product was dissolved in a small amount of DCM and added to Et₂O. A precipitate was formed and ether was removed. The remaining solid was washed a few times with Et₂O.

Yield: 29%

MS (ESI) m/z 607.9 [M+H]²⁺

¹H-NMR (MeOD, 400 MHz): δ 1.32 (m, 18H), 1.52 (m, 2H), 1.87 (m, 2H), 2.33 (t, J=7.2 Hz, 2H), 3.10 (m, 1H), 3.41 (m, 8H), 3.50 (m, 1H), 3.63 (m, 2H), 3.69 (m, 8H), 4.11 (m, 2H), 4.37 (m, 2H), 4.64 (m, 3H), 6.99 (d, J=2.4 Hz, 2H), 7.10 (dd, J=2.4 Hz and J=9.6 Hz, 2H), 7.17-7.44 (m, 16H), 7.54 (m, 1H), 7.73 (m, 1H), 7.80 (m, 2H), 7.94 (br s, 1H)

LC-MS tᵣ 15.5 min (90.8%)

The following compounds were prepared in a similar way:

Final compound 2: 2,2,2-trifluoroacetic acid, N-(6-(diethylamino)-9-(2-(4-(8-(4-(11-(diphenoxy phosphoryl)-12-(4-guanidinophenyl)-9-oxo-2,5,8-trioxa-10-azadodecyl)-1H-1,2,3-triazol-1-yl)octanoyl)piperazine-1-carbonyl)phenyl)-3H-xanthen-3-ylidene)-N-ethylethanaminium chloride

Yield: 66%

MS (ESI) m/z 629.9 [M+H)]²⁺

HPLC (214 nm) tᵣ 23.8 min (91.1%)

¹H-NMR (MeOD, 400 MHz) δ 1.32 (m, 18H), 1.53 (m, 2H), 1.89 (m, 2H), 2.33 (m, 2H), 3.12 (m, 1H), 3.39 (m, 8H), 3.62 (m, 6H), 8.37 (q, J=6.8 Hz, 8H), 4.09 (m, 2H), 4.38 (m, 2H), 4.62 (m, 2H), 4.67 (m, 1H), 6.9-8.0 (m, 25H)

LC-MS tᵣ 16.0 min (96.0%)

Final compound 3: 2,2,2-trifluoroacetic acid, N-(6-(diethylamino)-9-(2-(4-(8-(4-(14-(diphenoxy phosphoryl)-15-(4-guanidinophenyl)-12-oxo-2,5,8,11-tetraoxa-13-azapentadecyl)-1H-1,2,3-triazol-1-yl)octanoyl)piperazine-1-carbonyl)phenyl)-3H-xanthen-3-ylidene)-N-ethylethanaminium chloride

Extra purification steps: product was dissolved in DCM and added to EtOAc. A precipitate was formed. This procedure was repeated, but the compound was added to hexane.

Yield: 37%

MS (ESI) m/z 651.9 [M+H]²+1H-NMR (MeOD, 400 MHz)_{—}1.30 (m, 18H), 1.52 (m, 2H), 1.87 (m, 2H), 2.32 (t, J=7.2 Hz, 2H), 3.10 (m, 1H), 3.98 (m, 8H), 3.56-3.71, (m, 18H), 4.07 (m, 2H), 4.36 (m, 2H), 4.60 (m, 3H), 6.95 (d, J=2.4 Hz, 2H), 7.06 (dd, J=2.4 Hz and J=9.6 Hz, 2H), 7.15-7.52 (m, 16H), 7.69 (m, 1H), 7.77 (m, 2H), 7.90 (m, 2H)

LC-MS tᵣ 13.9 min (90.1%)

Final compound 4: 2,2,2-trifluoroacetic acid, N-(6-(diethylamino)-9-(2-(4-(8-(4-(3-(((1-(diphenoxyphosphoryl)-2-(4-guanidinophenyl)ethyl)carbamoyl)oxy)propyl)-1H-1,2,3-triazol-1-yl)octanoyl)piperazine-1-carbonyl)phenyl)-3H-xanthen-3-ylidene)-N-ethylethanaminium chloride

Yield: 65%

MS (ESI) m/z 599.8 [M+H]²⁺

¹H-NMR (MeOD, 400 MHz) δ 1.30 (m, 18H), 1.52 (m, 2H), 1.86 (m, 4H), 2.32 (m, 2H), 2.69 (m, 2H), 3.10 (m, 1H), 3.38 (m, 8H), 3.68 (q, J=6.8 Hz, 8H), 3.98 (m, 2H), 4.33 (m, 2H), 4.63 (m, 1H), 6.9-7.8 (m, 25H)

HPLC (214 nm) tᵣ 23.8 min (91.8%)

LC-MS tᵣ 16.4 min (97.1%)

Final compound 5: 2,2,2-trifluoroacetic acid, N-(9-(2-(4-(8-(4-(3-(((1-(bis(4-acetamidophenoxy)phosphoryl)-2-(4-guanidinophenyl)ethyl)carbamoyl)oxy)propyl)-1H-1,2,3-triazol-1-yl)octanoyl)piperazine-1-carbonyl)phenyl)-6-(diethylamino)-3H-xanthen-3-ylidene)-N-ethylethanaminium chloride

Yield: 51%

MS (ESI) m/z 657.2 [M+H]²⁺

¹H-NMR (MeOD, 400 MHz) δ 1.34 (m, 18H), 1.51 (m, 2H), 1.85 (m, 4H), 2.11 (s, 6H), 2.31 (t, 2H), 2.68 (t, J=7.6 Hz, 2H), 3.13 (m, 1H), 3.36 (m, 9H), 3.65 (q, 8H), 3.95 (m, 2H), 4.30 (t, 2H), 4.60 (m, 1H), 6.94-7.55 (m, 23H)

LC-MS tᵣ 14.5 min (91.3%)

Final Compound 6: 10-(7-(4-(3-(((1-(diphenoxyphosphoryl)-2-(4-guanidinophenyl)ethyl)carbamoyl) oxy)propyl)-1-1,2,3-triazol-1-yl)heptyl)-5,5-difluoro-1,3,7,9-tetramethyl-5H-dipyrrolo[1,2-c:2′,1′-f][1,3,2]diazaborinin-4-ium-5-uide 2,2,2-trifluoroacetate

THF was used instead of t-BuOH. No precipitation as purification step. Product was pure after extraction.

Yield: 82%

MS (ESI) m/z 908.5 [M+1]⁺

¹H-NMR (MeOH, 400 MHz): δ 1.25-1.65 (m, 8H), 1.88 (m, 4H), 2.43 (s, 6H), 2.45 (s, 6H), 2.68 (t, J=7.5 Hz, 2H), 3.00 (m, 2H), 3.09 (m, 1H), 3.43 (m, 1H), 3.97 (t, J=6.1 Hz, 2H), 4.34 (t, J=6.9 Hz, 2H), 4.64 (m, 1H), 6.13 (s, 2H), 7.15-7.50 (m, 14H ), 7.67 (s, 1H)

LC-MS tᵣ 17.7 min (95.0%)

### Reaction Procedure W

### Final compound 7: 3-(1-(13-oxo-17-((3aS,4S,6aR)-2-oxohexahydro-1H-thieno[3,4-d]imidazol-4-yl)-3,6,9-trioxa-12-azaheptadecyl)-1H-1,2,3-triazol-4-yl)propyl

(1-(diphenoxyphosphoryl)-2-(4-guanidinophenyl)ethyl)carbamate 2,2,2-trifluoroacetatepent-4-yn-1-yl (1-(diphenoxyphosphoryl)-2-(4-(2,3-bis(tert-butoxycarbonyl)guanidine)phenyl)ethyl)carbamate (intermediate 49) (0.139 mmol) and N-(2-(2-(2-(2-azidoethoxy)ethoxy)ethoxy)ethyl)-5-((3aS,4S,6aR)-2-oxohexahydro-1H-thieno[3,4-d]imidazol-4-yl)pentanamide (intermediate 72) (0.139 mmol) were dissolved in a mixture of t-BuOH and H₂O (600 μl each) followed by the addition of sodium ascorbate (0.333 mmol) and CuSO₄ (0.083 mmol). The resulting solution was allowed to stir for 4 hours at room temperature. Water and DCM were added and the water layer was washed 2 more times with DCM. The combined organic layers were washed with 1N HCl, saturated bicarbonate and brine solution, dried over anhydrous Na₂SO₄, filtered and evaporated. The resulting crude mixture was purified by flash chromatography (20% MeOH in EtOAc) to obtain 3-(1-(13-oxo-17-((3aS,4S,6aR)-2-oxohexahydro-1H-thieno[3,4-d]imidazol-4-yl)-3,6,9-trioxa-12-azaheptadecyl)-1H-1,2,3-triazol-4-yl)propyl (1-(diphenoxyphosphoryl)-2-(4-(2,3-bis(tert-butoxycarbonyl)guanidinophenyl)ethyl)carbamate 2,2,2-trifluoroacetate

Yield: 62%

MS (ESI) m/z 1187.8 [M+Na]⁺

¹H-NMR (MeOD, 400 MHz): δ 1.45 (s, 9H), 1.51 (m, 2H), 1.57 (s, 9H), 1.60-1.75 (m, 4H), 1.88 (m, 2H), 2.18 (t, J=7.4 Hz, 2H), 2.67 (m, 3H), 2.90 (dd, 1H), 3.05 (m, 1H), 3.17 (m, 1H), 3.40 (m, 1H), 3.50 (m, 2H), 3.55 (s, 4H), 3.58 (s, 4H), 3.85 (t, J=5.1 Hz, 2H), 3.96 (dt, J=1.7 Hz and 6.1 Hz, 2H), 4.26 (m, 1H), 4.46 (m, 1H), 4.51 (t, J=4.6 Hz, 2H), 4.64 (m, 1H), 7.1-7.55 (m, 14H), 7.76 (s, 1H)

This obtained “click” product was deprotected by dissolving it in 1 ml DCM and 1 ml TFA. The solution was stirred for 2 hours at room temperature, before the solvent was evaporated. Cold ether was added to wash the compound.

Yield: 54%

MS (ESI) m/z 965.5 [M+1]⁺, 494.1 [(M+Na)/2]⁺

¹H-NMR (MeOD, 400 MHz): δ 1.44 (m, 2H), 1.50-1.75 (m, 4H), 1.89 (m, 2H), 2.20 (t, J=7.4 Hz, 2H), 2.70 (m, 3H), 2.92 (dd, 1H), 3.09 (m, 1H), 3.19 (m, 1H), 3.44 (m, 1H), 3.51 (m, 2H), 3.58 (s, 4H), 3.61 (s, 4H), 3.89 (t, J=5.1 Hz, 2H), 4.00 (m, 2H), 4.29 (m, 1H), 4.48 (m, 1H), 4.51 (t, J=4.9 Hz, 2H), 4.64 (m, 1H), 7.15-7.50 (m, 14H), 7.76 (s, 1H)

LC-MS tᵣ 12.9 min (98.2%)

The following compounds were prepared in a similar way:

### Final compound 8: 3-(1-(1-(4-fluorophenyl)-1-oxo-5,8,11-trioxa-2-azamidecan-13-yl)-1H-1,2,3-triazol-4-yl)propyl (1-(diphenoxyphosphoryl)-2-(4-guanidinophenyl)ethyl)carbamate hydrochloride

Instead of t-BuOH, THF was used. Flash purification (10% MeOH in EtoAc)

After deprotection of the “click” product, the obtained compound was converted to a HCl salt by adding 1N HCl in ether to the TFA salt. A precipitate was formed.

Yield: 98%

¹H-NMR (MeOD, 400 MHz): δ 1.95 (m, 2H), 2.80 (t, J=8.0 Hz, 2H), 3.10 (m, 1H), 3.41 (m, 1H), 3.56 (m, 12H), 3.90 (t, J=8.0 Hz, 2H), 3.99 (m, 1H), 4.04 (m, 1H), 4.65 (m, 3H), 7.20 (m, 10H), 7.38 (m, 4H), 7.45 (d, J=8.0 Hz, 2H), 7.88 (dd, J=5.3 Hz and J=7.2 Hz, 2H), 8.21 (s, 1H)

LC-MS tᵣ 14.1 min (97.4%)

Final compound 9: 2-((1E,3E,5E)-5-(1-(6-((2-(4-(3-(((1-(diphenoxyphosphoryl)-2-(4-guanidino phenyl)ethyl)carbamoyl)oxy)propyl)-1H-1,2,3-triazol-1-yl)ethyl)amino)-6-oxohexyl)-3,3-dimethyl indolin-2-ylidene)penta-1,3-dien-1-yl)-1,3,3-trimethyl-3H-indol-1-ium

Product was freeze-dried (dissolved in Water/t-BuOH 3/1)

Yield: 87%

¹H-NMR (MeOD, 400 MHz): 1.41 (m, 2H), 1.60 (m, 2H), 1.72 (m, 8H), 1.78 (m, 3H), 1.89 (m, 3H), 2.17 (t, J=7.2 Hz, 2H), 2.70 (t, J=7.6 Hz, 2H), 3.11 (m, 1H), 3.37 (s, 2H), 3.45 (m, 1H), 3.62 (s, 4H), 3.99 (t, J=6.0 Hz, 2H), 4.11 (t, J=7.6 Hz, 1H), 4.45 (t, J=5.6 Hz, 2H), 4.65 (m, 1H), 6.28 (dd, J=13.6 Hz and J=17.6 Hz), 2H), 6.63 (t, J=12.4 Hz, 1H), 7.15-7.52 (m, 22H), 7.70 (s, 1H), 8.04 (d, J=8.8H, 1H), 8.26 (t, J=11.6 Hz, 2H)

LC-MS tᵣ 17.4 min (92.2%)

### In Vitro and In Vivo Assays

uPA Inhibition: In Vitro Evaluation.

Enzymatic activity was measured at 37° C. in a Spectramax 340 (Molecular Devices) microtiter plate reader using the chromogenic substrate Biophen CS-61(44) (LpyroGlu-Gly L-Arg-p-NA.HCl), with a Km of 80 μM.

The substrate and human enzyme were obtained from Nodia. The reaction was monitored at 405 nm, and the initial rate was determined between 0 and 0.25 absorbance units in 20 min. The reaction mixture contained 250 μM substrate and approximately 1 mU of enzyme in 145 μL of buffer in a final volume of 200 μL. A 50 mM Tris buffer, pH 8.8, was used. From each inhibitor concentration, 5 μL was added, obtaining a final concentration from 0 to 250 μM in a total volume of 0.2 mL. Activity measurements were routinely performed in duplicate. The IC₅₀ value is defined as the concentration of inhibitor required to reduce the enzyme activity to 50% after a 15 min preincubation with the enzyme at 37° C. before addition of the substrate. IC₅₀ values were obtained by fitting the data with the four-parameter logistics equation using Grafit 5.

$$v = \frac{v range}{1 + {e}^{sln \vert {I}_{0} / {IC}_{50} \vert}} + background$$

where s=slope factor, v=rate, I₀=inhibitor concentration, and range=the fitted uninhibited value minus the background. The equation assumes the y falls with increasing x.

Inhibitor stock solutions were prepared in DMSO and stored at −20° C. Because the compounds described in this paper completely inactivate uPA following pseudofirst-order kinetics, the IC₅₀ value is inversely correlated with the second-order rate constant of inactivation. For a simple pseudofirst-order inactivation process, the activity after incubation with inhibitor (vᵢ) varies with the inhibitor concentration (i), as described in the following equation:

vᵢ=vₒ×e^{-k}^{ᵢ}t,

where vₒ is the activity in absence of inhibitor, k is the second-order rate constant of inactivation, and t is the time. The inactivation rate constant was determined from the time course of inhibition.

The inhibitor was mixed with the substrate (250 μM final concentration), and the buffer solution with the enzyme was added at the time zero. The inhibitor concentrations were chosen to obtain total inhibition of the enzyme within 20 min. The progress curves show the absorbance of p-nitroanilide produced as a function of time. Initially, no inhibitor is bound to the enzyme, and the tangent to the progress curve (dA/dt) is proportional to the concentration of the free enzyme. The concentration of free enzyme decreases over time due to the kinetics of inhibitor binding, as described above. Progress curves were recorded in pseudofirst-order conditions ([I]₀>>[E]₀) and with less than 10% conversion of the substrate during the entire time course. In these conditions, dA/dt decreases exponentially with time. The progress curves were fitted with the integrated rate equation to yield a value for k_{obs}, a pseudofirst-order rate constant

Aₜ=v_{0[}1−e^{-k}^{_{obs}}^{t}]/k_{obs}+A₀

where Aₜ=absorbance at time t, A₀=absorbance at time zero, and v_{0=uninhibited initial rate.}

The apparent second-order rate constant (kₐₚₚ) was calculated from the slope of the linear part of the plot of k_{obs} versus the inhibitor concentration ([I]₀). In case of competition between the inhibitor and the substrate, kₐₚₚ is smaller than the “real” second order rate constant k discussed above because a certain fraction of the enzyme is present as an enzyme-substrate complex. kₐₚₚ depends on the substrate concentration used in the experiment, as described by Lambeir et al.⁴⁰ For most final compounds, the k_{obs} versus [I]₀ plot was not linear, but could be fitted with a hyperbolic function: k_{obs}=k₂[I]₀/(K₁+[I]₀) (two step mechanism). Where K₁ is the dissociation constant of the reversible enzyme-inhibitor complex and k₂ is the first-order rate constant associated with irreversible modification of the enzyme. A kₐₚₚ could be calculated as k₂/K₁.

Determination of the Selectivity for uPA.

The IC₅₀ values for plasmin (from human plasma, Sigma), tPA (recombinant, Nodia), thrombin (from human plasma, Sigma), and FXa were determined in the same way as for uPA. Biophen CS-05(88) (H-D-Ile-Pro-Arg-pNa.2HCl) for tPA (Km: 1 mM), biophen CS-21(66) (pyroGlu-Pro-Arg-pNA.HCl) for plasmin (Km: 400 μM) and thrombin (Km: 150 μM), and Biophen CS-11(32) (Suc-Ile-Gly (ã Pip)Gly-Arg-pNA.HCl) for

FXa (Km: 1.5 mM) were used as substrates. The mixture contained 580 μM substrate for thrombin and plasmin, 1.25 mM for tPA, 522 μM for FXa, and 425 μM for trypsin, approximately 5 mU of enzyme, and 145 μL of buffer. For tPA and thrombin, Tris buffer, pH 8.3, was used, for FXa, Tris buffer, pH 8.3, for plasmin, Tris buffer, pH 7.4, was used. Results are shown in table 1.

TABLE 1
Compound Selectivity

| | | IC₅₀ (μM) | | | | |
|---|---|---|---|---|---|---|
| Compounds | uPA: kₐₚₚ (M⁻¹ s⁻¹) | uPA | tPA | Thrombin | Plasmin | FXa |

INTERMEDIATES

| | | | | | | |
|---|---|---|---|---|---|---|
| 55 | 19 × 10³ ± 4 × 10³ * | 0.0088 ± 0.0008 | 66 ± 13 | 33 ± 4 | 11 ± 1 | 50% @ 250 |
| 56 | 12 × 10³ ± 5 × 10³ * | 0.0085 ± 0.0012 | 65 ± 9 | 20 ± 3 | 7.8 ± 0.6 | 58% @ 250 |
| 57 | 11 × 10³ ± 4 × 10³ * | 0.011 ± 0.001 | 67 ± 7 | 21 ± 5 | 9 ± 2 | 54% @ 250 |
| 58 | 30 × 10³ ± 9 × 10³ * | 0.0080 ± 0.0005 | 29 ± 2 | 12.2 ± 0.3 | 3.4 ± 0.4 | 50% @ 250 |
| 59 | 14 × 10³ ± 3 × 10³ * | 0.0096 ± 0.0019 | 16.7 ± 1.7 | 2.4 ± 0.2 | 3.6 ± 0.3 | 61% @ 250 |
| 60 | 19 × 10³ ± 8 × 10³ * | 0.0052 ± 0.0010 | 56 ± 53 | 11.8 ± 0.6 | 7.9 ± 0.3 | 57% @ 250 |
| 61 | 10 × 10³ ± 4 × 10³ * | 0.015 ± 0.002 | 62 ± 8 | 13 ± 0.5 | 5.7 ± 0.6 | 51% @ 250 |
| 62 | 9 × 10³ ± 4 × 10³ * | 0.015 ± 0.002 | 70 ± 11 | 29 ± 5 | 9.0 ± 0.7 | 48% @ 250 |
| 63 | 40 × 10³ ± 8 × 10³ * | 0.0035 ± 0.0017 | 24 ± 4 | 2.1 ± 0.2 | 1.4 ± 0.1 | 70% @ 250 |

FINAL COMPOUNDS

| | | | | | | |
|---|---|---|---|---|---|---|
| 1 | 17 × 10² ± 1 × 10² | 0.017 ± 0.002 | 7.9 ± 0.3 | 7.9 ± 1.7 | 5.4 ± 0.6 | 11% @ 2.5 |
| 2 | 13.0 × 10² ± 0.7 × 10² | 0.019 ± 0.003 | 33 ± 13 | 24 ± 21 | 5.4 ± 1.6 | 176 ± 128 |
| 3 | 26 × 10² ± 2 × 10² | 0.030 ± 0.006 | 42 ± 5 | 13.8 ± 1.3 | 11 ± 4 | 39 ± 22 |
| 4 | 49 × 10² ± 0.9 × 10² | 0.025 ± 0.007 | 8 ± 1 | 3.5 ± 0.8 | 4.6 ± 2.3 | 65 ± 14 |
| 5 | 52 × 10² ± 1 × 10² * | 0.022 ± 0.002 | >62 | 5.2 ± 0.2 | 5.6 ± 1.5 | 90% @ 250 |
| 6 | 12.2 × 10² ± 0.5 × 10² * | 0.0467 ± 0.0015 | 19% I @ 250 | ±125 | 0.674 ± 0.123 | 19% I @ 250 |
| 7 | 86 × 10² ± 10 × 10² * | 0.0156 ± 0.0010 | 58 ± 6 | 14.1 ± 0.3 | 4.7 ± 0.1 | 73% I @ 250 |
| 8 | 68 × 10² ± 25 × 10² * | 0.019 ± 0.002 | 46 ± 5 | 13.2 ± 0.6 | 5.9 ± 0.8 | ≈62 |
| 9 | 16 × 10² ± 1 × 10² | 0.0589 ± 0.0017 | | >2.5 | >2.5 | |

* Compound showed a two-step mechanism, kₐₚₚ calculated as k₂/K₁

### REFERENCES

1. Martin, P. M.; Dussert, C.; Romain, S.; Ouafik, L. H. The plasmin/plasminogen system and cancer. Oncologie 2010, 12, 322-340.
2. Oikonomopoulou, K.; Diamandis, E. P.; Hollenberg, M. D. Kallikrein-related peptidases: proteolysis and signaling in cancer, the new frontier. Biological Chemistry 2010, 391, 299-310.
3. Sanders, A. J.; Webb, S. L.; Parr, C.; Mason, M. D.; Jiang, W. G. The Type II Transmembrane Serine Protease, Matriptase-2: Possible Links to Cancer? Anti-Cancer Agents in Medicinal Chemistry 2010, 10, 64-69.
4. Sotiropoulou, G.; Pampalakis, G. Kallikrein-related peptidases: bridges between immune functions and extracellular matrix degradation. Biological Chemistry 2010, 391, 321-331.
5. Lund, L. R.; Green, K. A.; Stoop, A. A.; Ploug, M.; Almholt, K.; Lilla, J.; Nielsen, B. S.; Christensen, I. J.; Craik, C. S.; Werb, Z.; Dano, K.; Romer, J. Plasminogen activation independent of uPA and tPA maintains wound healing in gene-deficient mice. Embo Journal 2006, 25, 2686-2697.
6. Milner, J. M.; Patel, A.; Davidson, R. K.; Swingler, T. E.; Desilets, A.; Young, D. A.; Kelso, E. B.; Donell, S. T.; Cawston, T. E.; Clarke, I. M.; Ferrell, W. R.; Plevin, R.; Lockhart, J. C.; Leduc, R.; Rowan, A. D. Matriptase Is a Novel Initiator of Cartilage Matrix Resorption in Osteoarthritis. Osteoarthritis and Cartilage 2010, 18, S9-S10.
7. Glas, M.; Popp, B.; Angele, B.; Koedel, U.; Chahli, C.; Schmalix, W. A.; Anneser, J. M.; Pfister, H. W.; Lorenzl, S. A role for the urokinase-type plasminogen activator system in amyotrophic lateral sclerosis. Experimental Neurology 2007, 207, 350-356.
8. Bahgat, M. M.; Blazejewska, P.; Schughart, K. Inhibition of lung serine proteases in mice: a potentially new approach to control influenza infection. Virology Journal 2011, 8, -.
9. Antalis, T. M.; Buzza, M. S.; Hodge, K. M.; Hooper, J. D.; Netzel-Arnett, S. The cutting edge: membrane-anchored serine protease activities in the pericellular microenvironment. Biochemical Journal 2010, 428, 325-346.
10. Duffy, M. J. The urokinase plasminogen activator system: Role in malignancy. Current Pharmaceutical Design 2004, 10, 39-49.
11. Ulisse, S.; Baldini, E.; Sorrenti, S.; D'Armiento, M. The Urokinase Plasminogen Activator System: A Target for Anti-Cancer Therapy. Current Cancer Drug Targets 2009, 9, 32-71.
12. Dass, K.; Ahmad, A.; Azmi, A. S.; Sarkar, S. H.; Sarkar, F. H. Evolving role of uPA/uPAR system in human cancers. Cancer Treat Rev 2008, 34, 122-36.
13. Kjellman, A.; Akre, O.; Gustafsson, O.; Hoyer-Hansen, G.; Lilja, H.; Norming, U.; Piironen, T.; Tornblom, M. Soluble urokinase plasminogen activator receptor as a prognostic marker in men participating in prostate cancer screening. Journal of Internal Medicine 2011, 269, 299-305.
14. Harbeck, N.; Thomssen, C. A New Look at Node-Negative Breast Cancer. Oncologist 2011, 16, 51-60.
15. Harbeck, N.; Schmitt, M.; Meisner, C.; Friedel, C.; Untch, M.; Schmidt, M.; Lisboa, B.; Sweep, C.; Janicke, F.; Thomssen, C.; Grp, C. N. S. Final 10-year analysis of prospective multicenter Chemo NO trial for validation of ASCO-recommended biomarkers uPA/PAI-1 for therapy decision making in node-negative breast cancer. Journal of Clinical Oncology 2009, 27, -.
16. Chu, S.C.; Yang, S. F.; Lue, K. H.; Hsieh, Y. S.; Hsiao, T. Y.; Lu, K. H. Urokinase-type plasminogen activator, receptor, and inhibitor correlating with gelatinase-B (MMP-9) contribute to inflammation in Gouty arthritis of the knee. Journal of Rheumatology 2006, 33, 311-317.
17. Das, A.; Boyd, N.; Jones, T. R.; Talarico, N.; McGuire, P. G. Inhibition of choroidal neovascularization by a peptide inhibitor of the urokinase plasminogen activator and receptor system in a mouse model. Archives of Ophthalmology 2004, 122, 1844-1849.
18. Fish, P. V.; Barber, C. G.; Brown, D. G.; Butt, R.; Collis, M. G.; Dickinson, R. P.; Henry, B. T.; Horne, V. A.; Huggins, J. P.; King, E.; O'Gara, M.; McCleverty, D.; McIntosh, F.; Phillips, C.; Webster, R. Selective urokinase-type plasminogen activator inhibitors. 4. 1-(7-Sulfonamidoisoquinolinyl)guanidines. Journal of Medicinal Chemistry 2007, 50, 2341-2351.
19. Iyer, A. M.; Zurolo, E.; Boer, K.; Baayen, J. C.; Giangaspero, F.; Arcella, A.; Di Gennaro, G. C.; Esposito, V.; Spliet, W. G. M.; Van Rijen, P. C.; Troost, D.; Gorter, J. A.; Aronica, E. Tissue Plasminogen Activator and Urokinase Plasminogen Activator in Human Epileptogenic Pathologies. Neuroscience 2010, 167, 929-945.
20. Annecke, K.; Schmitt, M.; Euler, U.; Zerm, M.; Paepke, D.; Paepke, S.; von Minckwitz, G.; Thomssen, C.; Harbeck, N. uPA and PAI-1 in breast cancer: Review of their clinical utility and current validation in the prospective NNBC-3 trial. Advances in Clinical Chemistry, Vol 45 2008, 45, 31-45.
21. Schmitt, M.; Mengele, K.; Napieralski, R.; Magdolen, V.; Reuning, U.; Gkazepis, A.; Sweep, F.; Brunner, N.; Foekens, J.; Harbeck, N. Clinical utility of level-of-evidence-1 disease forecast cancer biomarkers uPA and its inhibitor PAI-1. Expert Review of Molecular Diagnostics 2010, 10, 1051-1067.
22. Cravatt, B. F.; Wright, A. T.; Kozarich, J. W. Activity-based protein profiling: From enzyme chemistry. Annual Review of Biochemistry 2008, 77, 383-414.
23. Blum, G.; Weimer, R. M.; Edgington, L. E.; Adams, W.; Bogyo, M. Comparative Assessment of Substrates and Activity Based Probes as Tools for Non-Invasive Optical Imaging of Cysteine Protease Activity. Plos One 2009, 4, -.
24. Frye, S. V. The art of the chemical probe. Nature Chemical Biology 2010, 6, 159-161.
25. Kodadek, T. Rethinking screening. Nature Chemical Biology 2010, 6, 162-165.
26. Simon, G. M.; Cravatt, B. F. Activity-based Proteomics of Enzyme Superfamilies: Serine Hydrolases as a Case Study. Journal of Biological Chemistry 2010, 285, 11051-11055.
27. Bachovchin, D. A.; Ji, T. Y.; Li, W. W.; Simon, G. M.; Blankman, J. L.; Adibekian, A.; Hoover, H.; Niessen, S.; Cravatt, B. F. Superfamily-wide portrait of serine hydrolase inhibition achieved by library-versus-library screening. Proceedings of the National Academy of Sciences of the United States of America 2010, 107, 20941-20946.
28. Edgington, L. E.; Berger, A. B.; Blum, G.; Albrow, V. E.; Paulick, M. G.; Lineberry, N.; Bogyo, M. Noninvasive optical imaging of apoptosis by caspase-targeted activity-based probes. Nature Medicine 2009, 15, 967-U177.
29. Brown, C. M.; Ray, M.; Eroy-Reveles, A. A.; Egea, P.; Tajon, C.; Craik, C. S. Peptide Length and Leaving-Group Sterics Influence Potency of Peptide Phosphonate Protease Inhibitors. Chemistry & Biology 2011, 18, 48-57.
30. Rao, J. H.; Dragulescu-Andrasi, A.; Yao, H. Q.; Yao, H. Q. Fluorescence imaging in vivo: recent advances. Current Opinion in Biotechnology 2007, 18, 17-25.
31. Mahmood, U.; Weissleder, R. Near-infrared optical imaging of proteases in cancer. Molecular Cancer Therapeutics 2003, 2, 489-496.
32. Elias, D. R.; Thorek, D. L.; Chen, A. K.; Czupryna, J.; Tsourkas, A. In vivo imaging of cancer biomarkers using activatable molecular probes. Cancer Biomark 2008, 4, 287-305.
33. Oikonomopoulou, K.; Hansen, K. K.; Baruch, A.; Hollenberg, M. D.; Diamandis, E. P. Immunofluorometric activity-based probe analysis of active KLK6 in biological fluids. Biological Chemistry 2008, 389, 747-756.
34. Pan, Z. Y.; Jeffery, D. A.; Chehade, K.; Beltman, J.; Clark, J. M.; Grothaus, P.; Bogyo, M.; Baruch, A. Development of activity-based probes for trypsin-family serine proteases. Bioorganic & Medicinal Chemistry Letters 2006, 16, 2882-2885.
35. Martin, S. L.; Walker, B. 2011.
36. Law, B.; Curino, A.; Bugge, T. H.; Weissleder, R.; Tung, C. H. Design, synthesis, and characterization of urokinase plasminogen-activator-sensitive near-infrared reporter. Chemistry & Biology 2004, 11, 99-106.
37. Joossens, J.; Ali, O. M.; EI-Sayed, I.; Surpateanu, G.; Van der Veken, P.; Lambeir, A. M.; Setyono-Han, B.; Foekens, J. A.; Schneider, A.; Schmalix, W.; Haemers, A.; Augustyns, K. Small, potent, and selective diaryl phosphonate inhibitors for urokinase-type plasminogen activator with in vivo antimetastatic properties. Journal of Medicinal Chemistry 2007, 50, 6638-6646.
38. Van der Veken, P.; El Sayed, I.; Joossens, J.; Stevens, C. V.; Augustyns, K.; Haemers, A. Lewis acid catalyzed synthesis of N-protected diphenyl 1-aminoalkylphosphonates. Synthesis-Stuttgart 2005, 634-638.
39. Nguyen, T.; Francis, M. B. Practical synthetic route to functionalized rhodamine dyes. Org Lett 2003, 5, 3245-8.
40. Lambeir, A. M.; Borloo, M.; DeMeester, I.; Belyaev, A.; Augustyns, K.; Hendriks, D.; Scharpe, S.; Haemers, A. Dipeptide-derived diphenyl phosphonate esters: Mechanism-based inhibitors of dipeptidyl peptidase IV. Biochimica Et Biophysica Acta-General Subjects 1996, 1290, 76-82.

1. A compound represented by Formula I or a stereoisomer, tautomer, racemic, metabolite, pro- or predrug, salt, hydrate, or solvate thereof comprising: 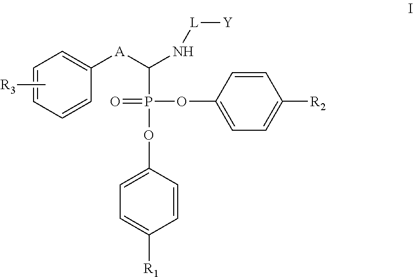
Wherein
R₁ and R₂ are each independently selected from the group consisting of —H, OH, -halo, C₁₋₆alkyl, —O—C₁₋₆alkyl, S—C₁₋₆alkyl, —NR₅R₆, —(C═O)—R₇, and SO₂—R₈;
R₅, R₆, R₉ and R₁₀ are each independently selected from the group consisting of —H, —O, C₁₋₆alkyl, —O—C₁₋₆alkyl, S—C₁₋₆alkyl, and —(C═O)—C₁₋₆alkyl;
R₇ and R₈ are each independently selected from the group consisting of -halo, —OH, C₁₋₆alkyl, —O—C₁₋₆alkyl, S—C₁₋₆alkyl, and —NR₉R₁₀;
R₃ is -guanidino;
A is selected from the group consisting of a direct bond, and C₁₋₆alkyl;
L is selected from the group consisting of —SO₂—R₄-amide-; —SO₂—R₄-sulphonamide; —SO₂—R₄-triazole-; —SO₂—R₄-urea-; —SO₂—R₄-amine; —SO₂—R₄-carbamate-; —(C═O)—R₄-amide-; —(C═O)—R₄-sulphonamide-; —(C═O)—R₄-triazole-; —(C═O)—R₄-urea-; —(C═O)—R₄-amine-; —(C═O)—R₄-carbamate-; —(C═O)—O—R₄-amide-; —(C═O)—O—R₄-sulphonamide-; —(C═O)—O—R₄-triazole-; —(C═O)—O—R₄-urea-; —(C═O)—O—R₄-amine-; —(C═O)—O—R₄-carbamate-; —(C═O)—N—R₄-amide-, —(C═O)—N—R₄-sulphonamide-; —(C═O)—N—R₄-triazole-; —(C═O)—N—R₄-urea-; —(C═O)—N—R₄-amine-; —(C═O)—N—R₄-carbamate-; —R₄-amide-, —R₄-sulphonamide-; —R₄-triazole-; —R₄-urea-; —R₄-amine-; —R₄-carbamate-;
R₄ is selected from the group consisting of —(CH₂)ₙ—, —(C₁₋₄alkyl-O)ₘ—, or —(C₁₋₄alkyl-O)ₘ—(CH₂)ₒ—;
m, n and o are each independently 1, 2, 3, 4, 5, 6, 7, 8, 9 or 10; and
Y represents a detectable label.

2. A compound according to claim 1 wherein
R₁ and R₂ are each independently selected from the group consisting of —H and —NH—(C═O)—CH₃;
R₃ is guanidino and is at the para position;
L is —(C═O)—O—R₄-triazole- or —(C═O)—R₄-triazole-;
R₄ is selected from the group consisting of —(CH₂)ₙ—, —(C₁₋₄alkyl-O)ₘ—, or —(C₁₋₄alkyl-O)ₘ—(CH₂)ₒ—;
m is 1, 2, 3, or 4;
n is 1, 2, 3, 4, 5, 6, 7, 8, 9 or 10;
o is 1, 2, 3, or 4; and
Y represents a detectable label.

3. A compound according to claim 1 wherein the detectable label can be instrumentally detected by magnetic resonance imaging, X-ray imaging, ultrasound, nuclear medicine imaging, multimodal imaging, fluorescence imaging, bioluminescence imaging, microscopy, mass detectors, wave length detectors, phosphorescent imaging, or chemiluminescent imaging.

4. A compound according to claim 1, wherein the detectable label is selected from radio-isotopes, fluorophores, imaging agents for MRI, X-ray responsive agents, and biotin labels or derivatives thereof.

5. An intermediate compound for preparing a compound according to claim 1 represented by Formula II: 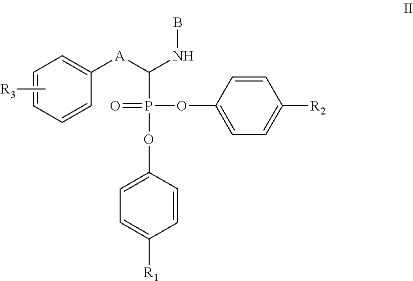
Wherein
R₁ and R₂ are each independently selected from the group consisting of —H, OH, -halo, C₁₋₆alkyl, —O—C₁₋₆alkyl, S—C₁₋₆alkyl, —NR₅R₆, —(C═O)—R₇, and SO₂—R₈;
R₅, R₆, R₉ and R₁₀ are each independently selected from the group consisting of —H, —O, C₁₋₆alkyl, —O—C₁₋₆alkyl, S—C₁₋₆alkyl, and —(C═O)—C₁₋₆alkyl;
R₇ and R₈ are each independently selected from the group consisting of -halo, —OH, C₁₋₆alkyl, —O—C₁₋₆alkyl, S—C₁₋₆alkyl, and —NR₉R₁₀;
R₃ is -guanidino;
A is selected from a direct bond and C₁₋₆alkyl;
B is selected from the group consisting of —(C═O)—O—R₄-alkyne, —(C═O)—O—R₄—N₃, —(C═O)—R₄-alkyne, and —(C═O)—R₄—N₃;
R₄ is selected from the group consisting of —(CH₂)ₙ—, —(C₁₋₄alkyl-O)ₘ—, or —(C₁₋₄alkyl-O)ₘ—(CH₂)ₒ—; and
m, n and o are each independently 1, 2, 3, 4, 5, 6, 7, 8, 9 or 10.

6. An intermediate compound according to claim 5, wherein
R₁ and R₂ are each independently selected from the group consisting of —H and —NH—(C═O)—CH₃;
R₃ is guanidino and is at the para position;
B is selected from the group consisting of —(C═O)—O—R₄-alkyne, —(C═O)—O—R₄—N₃, —(C═O)—R₄-alkyne-, or —(C═O)—R₄—N₃;
R₄ is selected from the group consisting of —(CH₂)ₙ—, —(C₁₋₄alkyl-O)ₘ—, or —(C₁₋₄alkyl-O)ₘ—(CH₂)ₒ—;
m is 1, 2, 3, or 4;
n is 1, 2, 3, 4, 5, 6, 7, 8, 9 or 10; and
o is 1, 2, 3, or 4.

7. (canceled)

8. (canceled)

9. (canceled)

10. (canceled)

11. (canceled)

12. (canceled)

13. A method for visualizing cancer cells in a human or animal comprising administering to said human or animal a labeled compound as defined in claim 1 and detecting the signal produced by the labeled compound as an indication of the presence of cancer cells.

14. The method according to claim 13, wherein the cancer cells express a trypsin-like serine protease.

15. A method for visualizing an active trypsin-like serine protease in a species comprising administering to said species a labeled compound according to claim 1 and detecting the signal produced by the labeled compound as an indication of the presence of said active trypsin-like serine protease.

16. A method for monitoring the effect of a treatment for inhibiting a trypsin-like serine protease in a species comprising administering to said species, at different timepoints a labeled compound according to claim 1 and detecting the signal produced by the labeled compound; wherein a reduction of the produced signal over time is an indication that said treatment is effective.

17. The method according to claim 15 wherein the species is selected from the list comprising: protein containing material, cell lysates, cells, tissue lysates, tissues, animals and humans.

18. The method according to claim 14; wherein the trypsin-like serine protease is urokinase plasminogen activator (uPA).
